(19) United States
(12) Patent Application Publication
(10) Pub. No.: US 2025/0347684 A1
(43) Pub. Date: Nov. 13, 2025

(54) METABOLIC BIOMARKERS IN BLOOD SERUM FOR DIAGNOSIS OF ALZHEIMER'S DISEASE

(71) Applicant: Board of Regents, The University of Texas System, Austin, TX (US)

(72) Inventors: Yingxin Zhao, Houston, TX (US); Miguel Angel Pappolla, Galveston, TX (US); Ernesto G. Miranda-Morales, Galveston, TX (US); Alejandro Villasante-Tezanos, Galveston, TX (US); Xiang Fang, Friendswood, TX (US)

(21) Appl. No.: 19/202,609

(22) Filed: May 8, 2025

Related U.S. Application Data
(60) Provisional application No. 63/646,072, filed on May 13, 2024.

(51) Int. Cl. G01N 33/53; A61B 5/00; G01N 33/68; G16H 20/10; G16H 50/20

(52) U.S. Cl. CPC G01N 33/5308; G01N 33/6848; G01N 33/6896; G16H 20/10; G16H 50/20; A61B 5/4088; G01N 2800/2821

(57) ABSTRACT

Provided herein is a method for treating a human subject with Alzheimer's Disease (AD) having an AD metabolomic phenotype, the method comprising: obtaining or having obtained a blood sample from the human subject with Alzheimer's disease; measuring the levels of metabolites in the blood sample; applying an algorithm to the measured metabolite levels, the algorithm generating a metabolomic score based on a comparison of the measured metabolites levels to reference metabolites levels; identifying the human subject with Alzheimer's Disease as having an AD metabolomic phenotype based on the metabolomic score; wherein the algorithm is selected from a machine learning algorithm, a clustering algorithm, a random forest algorithm, a support vector machines algorithm, a radial basis function algorithm and a combination thereof.

### CROSS-REFERENCE TO RELATED APPLICATIONS

This application claims priority to U.S. Provisional Application Ser. No. 63/646,072, filed May 13, 2024, the entire contents of which are incorporated herein by reference.

### STATEMENT OF FEDERALLY FUNDED RESEARCH

This invention was made with government support under AG066060 awarded by the National Institutes of Health. The government has certain rights in this invention.

### TECHNICAL FIELD

The present invention relates in general to the field of metabolic biomarkers in blood serum for diagnosis of Alzheimer's Disease (AD), and more particularly, to novel biomarkers in blood serum for diagnosis of Alzheimer's Disease.

### INCORPORATION-BY-REFERENCE OF MATERIALS FILED ON COMPACT DISC

Not applicable.

### BACKGROUND

Without limiting the scope of the disclosure, its background is described in connection with biomarker tests for Alzheimer's Disease.

Alzheimer's disease (AD) is a leading cause of dementia, accounting for 60-80% of dementia cases^{[12]}. By 2050, the global prevalence of all-cause dementia is projected to reach 113 million^{[2]}. AD is neuropathologically characterized by the accumulation of hyperphosphorylated tau and aggregates of amyloid-beta peptide in the brain. Clinically, patients with AD experience gradual and progressive cognitive decline and disability. Most treatments available for AD offer only temporary symptomatic relief without modifying the course of the disease^{[3]}. Recently, emerging early-stage anti-amyloid antibody therapies have shown very modest positive effects on disease progression. The consensus opinion in the scientific community is underscoring the imperative for early detection and intervention. This emphasizes the need to identify reliable biomarkers, preferably blood-based, for AD.

The National Institute on Aging and Alzheimer's Association (NIA-AA) recognizes cerebrospinal fluid (CSF) proteins such as total Tau (T-Tau), phosphorylated Tau (p-Tau), and amyloid β (Aγ342/40) as key AD biomarkers^{[4,5]}. Blood tests, however, offer a more practical alternative to CSF tests, especially for frequent assessments in clinical trials^{[6]}. Some currently used blood biomarkers include amyloid, tau species, neurofilament light chain (NFL) and plasma glial fibrillary acidic protein (GFAP). Yet, establishing consistent blood biomarkers for AD has been challenging. The compromised blood-brain barrier in aging and AD suggests that blood metabolites might more accurately represent brain biochemical alterations^{[7-11]}. Research increasingly associates the potential contribution of metabolic dysregulation in AD^{[12,13]}. Conditions such as diabetes, obesity, and metabolic syndrome, along with lifestyle factors, including diet and sedentary behavior, contribute significantly to AD risk^{[4-17]}. Teruya and colleagues identified some metabolites in plasma associated with dementia, though the specificity of these biomarkers to AD remains unexplored^{[18]}.

Despite these advancements, a need remains for the detection of AD at its earliest stages through biomarkers. The prevailing view in the research community suggests that early interventions may yield the most promising therapeutic outcomes. As such, pinpointing the disease at its earliest stages through biomarkers becomes paramount.

### SUMMARY

As embodied and broadly described herein, an aspect of the present disclosure relates to a method for treating a human subject with Alzheimer's Disease (AD) having an AD metabolomic phenotype, the method comprising: obtaining or having obtained a blood sample from the human subject with AD; measuring one or more levels of metabolites in the blood sample, wherein the metabolites are selected from at least one of benzenoids, organoheterocyclic compounds, prenol lipids, sterol lipids, fatty acyls, lipids and lipid-like molecules, cyclohexenones, imidazoles, and aryl alkyl ketones; applying an algorithm to the one or more measured metabolite levels, the algorithm generating a metabolomic score based on a comparison of the one or more measured metabolites levels to reference metabolites levels; and identifying the human subject with AD as having an AD metabolomic phenotype based on the metabolomic score; wherein the algorithm is selected from a machine learning algorithm, a clustering algorithm, a random forest algorithm, a support vector machines algorithm, a radial basis function algorithm and a combination thereof; and administering to the human subject with the AD metabolomic score one or more agents that slow progression of AD. In one aspect, the machine learning algorithm is selected from at least one of: Random Forest, Support Vector Machines, and Radial Basis Function. In another aspect, the method further comprises administering the one or more agents is selected from at least one of: an antagonist of N-methyl-D-aspartate (NMDA) receptor subtype of the glutamate receptor, memantine, Cholinesterase inhibitors (ChEIs), donepezil, galantamine and rivastigmine, a monoclonal antibody against beta amyloid, lecanemab, anti-inflammatory drugs, NSAIDs, non-selective NSAIDs, selective NSA IDs, steroids, glucocorticoids, Immune Selective Anti-Inflammatory Derivatives (ImSAIDs), anti-TNF medications, anti-IL5 drugs, CRP-lowering agents, anti-apoptotic compounds, metal chelators, inhibitors of DNA repair, 3-amino-1-propanesulfonic acid (3APS), 1,3-propanedisulfonate (1,3PDS), secretase activators, beta- and gamma-secretase inhibitors, neurotransmitters, beta-sheet breakers, anti-inflammatory molecules, antipsychotics, cholinesterase inhibitor, a corticosteroid, an antibiotic, an antiviral agent, an anti-Tau antibody, semorinemab, BMS-986168, C2N-8E12, Gosuranemab, Tilavonemab, Zagotenemab, a Tau inhibitor, a Tau N-terminal binder, a Tau mid-domain binder, a fibrillar Tau binder, an anti-amyloid-beta (anti-Ap) antibody, bapineuzumab, solanezumab, aducanumab, gantenerumab, crenezumab, donanembab, lecanemab, an beta-amyloid aggregation inhibitor, an anti-BACE1 antibody, a BACE1 inhibitor, a monoamine depletory agent, an ergoloid mesylate, an anticholinergic antiparkinsonism agent, a dopaminergic antiparkinsonism agent, a tetrabenazine, a dimebolin, a homotaurine, or a serotonin receptor activity modulator. In another aspect, the blood sample is a whole blood or a plasma sample, and wherein one or more biomarkers are measured by at least one method selected from ultra-high-performance liquid chromatography-high resolution mass spectrometry (UPLC-HRMS), an immunoassay, an enzymatic activity assay, fluorescence detection, chemiluminescence detection, electrochemiluminescence detection and patterned array, antibody binding, fluorescence activated sorting, detectable bead sorting, antibody array, microarray, enzymatic array, receptor binding array, solid-phase binding array, liquid phase binding array, fluorescent resonance transfer, and radioactive labeling. In another aspect, the algorithm is selected from Support Vector Machine (SVM) and Random Forest (RF) algorithms. In another aspect, the algorithm is selected from Support Vector Machine (SVM) with a Radial Basis Function (RBF) kernel. In another aspect, the at least one of: benzenoids are selected from at least one of: benzene, m-cresol, ethylparaben, hexylbenzene, or ionene; N-Nonanoylglycine, delta-Dodecalactone, 2,6-Di-tert-butylbenzoquinone, and 3-Methyl-2-cyclohexen-1-one; the organoheterocyclic compounds are selected from at least one of: 2-furoic acid or isoquinoline; the prenol lipids are selected from at least one of: 2,6-di-tert-butylbenzoquinone, 2,6-Di-tert-butylbenzoquinone; or (R)-Carvone; the sterol lipid is androstenedione; the fatty acyls are selected from at least one of: traumatic acid, gamma-nonalactone; Decanoic acid; or Undecylenic acid; the lipids and lipid-like molecules, cyclohexenones, imidazoles and aryl alkyl ketones are selected from at least one of: Linalyl butyrate, 3-Methyl-2-cyclohexen-1-one, 1-methylimidazole, and gamma-Oxo-3-pyridinebutanal, respectively; the metabolites are selected from Hexylbenzene, Ionene, 3-Methyl-2-cyclohexen-1-one, γ-Oxo-3-pyridinebutanal, 2,6-D i-tert-butylbenzoquinone (DK3970000), and Androstenedione; or the metabolites are selected from at least one of: Traumatic acid, Hexylbenzene, Benzene, 2,6-D i-tert-butylbenzoquinone (DK3970000), Ethylparaben, M-Cresol, 3-Methyl-2-cyclohexen-1-one (3360), 1-methylimidazole, or γ-Oxo-3-pyridinebutanal. In another aspect, the metabolites are selected in the following order: N-nonanoylglycine, delta-dodecalactone, 2,6-di-tert-butylbenzoquinone, 3-methyl-2-cyclohexen-1-one, linalyl butyrate, γ-oxo-3-pyridinebutanal, lilac acetaldehyde, traumatic acid, homovanillin, 2-tridecenal, androstenedione, 2-acetyl-3,5-dimethylfuran, 3-oxopalmitic acid, and 8-hydroxy-5,6-octadienoic acid.

As embodied and broadly described herein, an aspect of the present disclosure relates to a method for treating a human subject with Alzheimer's Disease (AD) having an AD metabolomic phenotype, the method comprising: obtaining or having obtained a blood sample from the human subject with Alzheimer's disease; measuring one or more levels of metabolites in the blood sample, wherein the metabolites are selected from 1, 2, 3, 4, 5, 6, 7, 8, 9, 10, 11, 12, 13, 14, 15, 16, 17, 18, 19, or 20 metabolites selected from Hexylbenzene, N-Nonanoylglycine, Ionene, 2401/delta-Dodecalacton, 3360/3-Methyl-2-cyclohexen-1-one, (5Z,8Z)-5,8-Tetradecadienoic acid, FG7175000, γ-Oxo-3-pyridinebutanal, DK3970000/2,6-Di-tert-butylbenzoquinone, 3,4-Dimethyl-5-pentyl-2-furanpropanoic acid, Androstenedione, D L-carvone, Decanoic acid, 10-Undecenoic acid, MFCD00009868, 4-Vinylcyclohexene, 1-Phenyl-2-hexanone, 2781/Gamma-nonalactone, 2,2-Methylenebisfuran, Allylcyclohexane; applying an algorithm to the measured metabolite levels, the algorithm generating a metabolomic score based on a comparison of the measured metabolites levels to reference metabolites levels; identifying the human subject with Alzheimer's Disease as having an AD metabolomic phenotype based on the metabolomic score; wherein the algorithm is selected from a machine learning algorithm, a clustering algorithm, a random forest algorithm, a support vector machines algorithm, a radial basis function algorithm and a combination thereof. In one aspect, the machine learning algorithm is selected from at least one of: Random Forest, Support Vector Machines, and Radial Basis Function. In another aspect, the method further comprises administering a drug treatment to the human subject identified with the metabolomic score, wherein the drug treatment is selected from at least one of: an antagonist of N-methyl-D-aspartate (NMDA) receptor subtype of the glutamate receptor, memantine, Cholinesterase inhibitors (ChEIs), donepezil, galantamine and rivastigmine, a monoclonal antibody against beta amyloid, lecanemab, anti-inflammatory drugs, NSAIDs, non-selective NSAIDs, selective NSAIDs, steroids, glucocorticoids, Immune Selective Anti-Inflammatory Derivatives (ImSAIDs), anti-TNF medications, anti-IL5 drugs, C-reactive protein (CRP)-lowering agents, anti-apoptotic compounds, metal chelators, inhibitors of DNA repair, 3-amino-1-propanesulfonic acid (3APS), 1,3-propanedisulfonate (1,3PDS), secretase activators, beta- and gamma-secretase inhibitors, neurotransmitters, beta-sheet breakers, anti-inflammatory molecules, antipsychotics, cholinesterase inhibitor, a corticosteroid, an antibiotic, an antiviral agent, an anti-Tau antibody, semorinemab, BMS-986168, C2N-8E12, Gosuranemab, Tilavonemab, Zagotenemab, a Tau inhibitor, a Tau N-terminal binder, a Tau mid-domain binder, a fibrillar Tau binder, an anti-amyloid-beta (anti-Ap) antibody, bapineuzumab, solanezumab, aducanumab, gantenerumab, crenezumab, donanembab, lecanemab, an beta-amyloid aggregation inhibitor, an anti-BACE1 antibody, a BACE1 inhibitor, a monoamine depletory agent, an ergoloid mesylate, an anticholinergic antiparkinsonism agent, a dopaminergic antiparkinsonism agent, a tetrabenazine, a dimebolin, a homotaurine, or a serotonin receptor activity modulator. In another aspect, the blood sample is a whole blood or a plasma sample, and wherein one or more biomarkers are measured by at least one method selected from ultra-high-performance liquid chromatography-high resolution mass spectrometry (UPLC-HRMS), an immunoassay, an enzymatic activity assay, fluorescence detection, chemiluminescence detection, electrochemiluminescence detection and patterned array, antibody binding, fluorescence activated sorting, detectable bead sorting, antibody array, microarray, enzymatic array, receptor binding array, solid-phase binding array, liquid phase binding array, fluorescent resonance transfer, and radioactive labeling. In another aspect, the algorithm is selected from Support Vector Machine (SVM) and Random Forest (RF) algorithms or Support Vector Machine (SVM) with a Radial Basis Function (RBF) kernel. In another aspect, the metabolites are selected in the following order: N-nonanoylglycine, delta-dodecalactone, 2,6-di-tert-butylbenzoquinone, 3-methyl-2-cyclohexen-1-one, linalyl butyrate, γ-oxo-3-pyridinebutanal, lilac acetaldehyde, traumatic acid, homovanillin, 2-tridecenal, androstenedione, 2-acetyl-3,5-dimethylfuran, 3-oxopalmitic acid, and 8-hydroxy-5,6-octadienoic acid.

As embodied and broadly described herein, an aspect of the present disclosure relates to a method for treating a human subject with Alzheimer's Disease (AD) having an AD metabolomic phenotype, the method comprising: obtaining or having obtained a blood sample from the human subject with Alzheimer's disease; measuring a level of metabolites in the blood sample, wherein the metabolites are selected from 1, 2, 3, or 4, 5, or 6 metabolites selected from N-nonanoylglycine, delta-dodecalactone, 2,6-di-tert-butylbenzoquinone, 3-methyl-2-cyclohexen-1-one, linalyl butyrate, or γ-oxo-3-pyridinebutanal; applying an algorithm to the measured metabolite levels, the algorithm generating a metabolomic score based on a comparison of the measured metabolites levels to reference metabolites levels; identifying the human subject with Alzheimer's Disease as having an AD metabolomic phenotype based on the metabolomic score; and administering an anti-inflammatory a drug treatment to the identified human subject; wherein the algorithm is selected from a machine learning algorithm, a clustering algorithm, a random forest algorithm, a support vector machines algorithm, a radial basis function algorithm and a combination thereof; and wherein the drug treatment is selected from at least one of: an antagonist of N-methyl-D-aspartate (NMDA) receptor subtype of the glutamate receptor, memantine, Cholinesterase inhibitors (ChEIs), donepezil, galantamine, rivastigmine, a monoclonal antibody against beta amyloid, lecanemab, anti-inflammatory drugs, NSAIDs, non-selective NSAID s, selective NSAIDs, steroids, glucocorticoids, Immune Selective Anti-Inflammatory Derivatives (ImSAIDs), anti-TNF medications, anti-IL5 drugs, C-reactive protein (CRP)-lowering agents, anti-apoptotic compounds, metal chelators, inhibitors of DNA repair, 3-amino-1-propanesulfonic acid (3APS), 1,3-propanedisulfonate (1,3PDS), secretase activators, beta- and gamma-secretase inhibitors, neurotransmitters, beta-sheet breakers, anti-inflammatory molecules, antipsychotics, cholinesterase inhibitor, a corticosteroid, an antibiotic, an antiviral agent, an anti-Tau antibody, semorinemab, BMS-986168, C2N-8E12, Gosuranemab, Tilavonemab, Zagotenemab, a Tau inhibitor, a Tau N-terminal binder, a Tau mid-domain binder, a fibrillar Tau binder, an anti-amyloid-beta (anti-AP) antibody, bapineuzumab, solanezumab, aducanumab, gantenerumab, crenezumab, donanembab, lecanemab, an beta-amyloid aggregation inhibitor, an anti-BACE1 antibody, a BACE inhibitor, a monoamine depletory agent, an ergoloid mesylate, an anticholinergic antiparkinsonism agent, a dopaminergic antiparkinsonism agent, a tetrabenazine, a dimebolin, a homotaurine, or a serotonin receptor activity modulator. In one aspect, the method further comprises measuring one or more additional level of metabolites in the blood sample selected from at least one of: (5Z,8Z)-5,8-Tetradecadienoic acid, FG7175000, 3,4-Dimethyl-5-pentyl-2-furanpropanoic acid, DL-carvone, Decanoic acid, 10-Undecenoic acid, MFCD00009868, 4-Vinylcyclohexene, 1-Phenyl-2-hexanone, 2781/Gamma-nonalactone, 2,2-Methylenebisfuran, or Allylcyclohexane, Hexylbenzene, Ionene, and Androstenedione.

As embodied and broadly described herein, an aspect of the present disclosure relates to a kit for determining an Alzheimer's Disease (AD) metabolomic phenotype, the kit comprising: a substrate comprising reagents that binds to one or more metabolites selected from 1, 2, 3, 4, 5, or 6 metabolites selected from N-nonanoylglycine, delta-dodecalactone, 2,6-di-tert-butylbenzoquinone, 3-methyl-2-cyclohexen-1-one, linalyl butyrate, or γ-oxo-3-pyridinebutanal, wherein the substrate is adapted for quantitative metabolite analysis; and instructions for the quantitative metabolite analysis. In another aspect, the kit further comprises additional reagents for measuring one or more additional level of metabolites in a blood sample selected from at least one of: (5Z,8Z)-5,8-Tetradecadienoic acid, FG7175000, 3,4-Dimethyl-5-pentyl-2-furanpropanoic acid, DL-carvone, Decanoic acid, 10-Undecenoic acid, MFCD00009868, 4-Vinylcyclohexene, 1-Phenyl-2-hexanone, 2781/Gamma-nonalactone, 2,2-Methylenebisfuran, or Allylcyclohexane, Hexylbenzene, Ionene, and Androstenedione. In another aspect, the substrate is selected for use in multiplexed tandem mass spectrometry (MS/MS) or high-performance liquid chromatography (UHPLC)-MS/MS analysis. In another aspect, the substrate is at least one of: strip, particle, bead, biodegradable particle, sheet, gel, filter, membrane, nylon membrane, fiber, capillary, needle, microtiter strip, tube, plate, well, comb, pipette tip, micro array, chip, or slide.

As embodied and broadly described herein, an aspect of the present disclosure relates to a non-transitory computer-readable medium for determining an Alzheimer's Disease (AD) metabolomic score in a subject comprising instructions stored thereon, that when executed on a processor, perform the steps of: receiving an electronic communication containing data for one or more levels of metabolites in the blood sample, wherein the metabolites are selected from at least one of benzenoids, organoheterocyclic compounds, prenol lipids, sterol lipids, fatty acyls, lipids and lipid-like molecules, cyclohexenones, imidazoles, and aryl alkyl ketones of the subject; using a processor to process an algorithm to the data for one or more levels of metabolites, the algorithm generating a metabolomic score based on a comparison of the data for one or more levels of metabolites to reference metabolites levels, wherein the algorithm is selected from a machine learning algorithm, a clustering algorithm, a random forest algorithm, a support vector machines algorithm, a radial basis function algorithm and a combination thereof; identifying the human subject with Alzheimer's Disease as having an AD metabolomic phenotype based on the metabolomic score; and administering to the human subject with the AD metabolomic score one or more agents that slow progression of AD.

As embodied and broadly described herein, an aspect of the present disclosure relates to a computer-implemented method for determining a personalized risk assessment for a subject, the method comprising: receiving an electronic communication containing data for one or more levels of metabolites in the blood sample, wherein the metabolites are selected from at least one of benzenoids, organoheterocyclic compounds, prenol lipids, sterol lipids, fatty acyls, lipids and lipid-like molecules, cyclohexenones, imidazoles, and aryl alkyl ketones of the subject; using a processor to process an algorithm to the data for one or more levels of metabolites, the algorithm generating a metabolomic score based on a comparison of the data for one or more levels of metabolites to reference metabolites levels, wherein the algorithm is selected from a machine learning algorithm, a clustering algorithm, a random forest algorithm, a support vector machines algorithm, a radial basis function algorithm and a combination thereof; identifying the human subject with Alzheimer's Disease as having an AD metabolomic phenotype based on the metabolomic score; and administering to the human subject with the AD metabolomic score one or more agents that slow progression of AD.

### BRIEF DESCRIPTION OF THE DRAWINGS

For a more complete understanding of the features and advantages of the present disclosure, reference is now made to the detailed description of the invention along with the accompanying figures and in which:

FIG. 1 shows a workflow for identifying metabolic biomarkers for AD diagnosis. The study was conducted in two phases: the discovery phase and the verification phase. In each phase, serum metabolome from AD patients and healthy controls were analyzed with ultra-high performance liquid chromatography-high resolution mass spectrometry (UHPLC-HRMS). Then, the biomarker candidates were identified using machine learning and further verified in the verification phase.

FIGS. 2A to 2F show the identification of blood-based metabolites that differentiate AD and cognitively unimpaired older adults. (FIG. 2A) Volcano plot of metabolites quantified in the discovery study. Red, metabolites were significantly elevated in the serum of AD patients; Green, metabolites were significantly depleted in the serum of AD patients; Grey, non-significant metabolites. (FIG. 2B) Heatmap of significant metabolites identified in the discovery study. (FIG. 2C) Volcano plot of metabolites quantified in the discovery study. Red, metabolites were significantly elevated in the serum of AD patients; Green, metabolites were significantly depleted in the serum of AD patients; Grey, non-significant metabolites. (FIG. 2D) Heatmap of significant metabolites identified in the discovery study. (FIG. 2E) Venn diagram of the significant metabolites identified in the discovery and verification studies. (FIG. 2F) Comparison of the profiles of the significant metabolites identified in both discovery and verification studies.

FIGS. 3A to 3F show classification feature optimization. (FIG. 3A) Classification error rate versus the number of features used in the discovery study. (FIG. 3B) Principal component analysis (PCA) analysis of subjects in the discovery studies using the top 14 features identified by the Support vector machine (SVM) algorithm and 4-fold cross-validation. (FIG. 3C) Profiles of the top 14 metabolite features in the discovery study. (FIG. 3D) Profiles of the top 14 metabolite features in the verification study. (FIG. 3E) Classification error rate versus the number of features used in the verification study. (FIG. 3F) PCA analysis of subjects of the verification studies using the top 14 features identified in the discovery study.

FIG. 4 shows the performance of SVM classification model. Classification model with 14 metabolic features using the discovery dataset and tested with the verification dataset. The receiver operating characteristic curve (ROC) and area under the curve (AUC) of each individual metabolite and the combination of 14 features were shown.

FIGS. 5A and 5B show the performance of Random Forest (RF) classification model. (FIG. 5A) Prediction error rate of RF model. (FIG. 5B) AUC of ROCs of RF model.

FIG. 6 is a graph that shows the performance of SVM classification model using endogenous metabolites. The AUC of ROCs and the prediction error rates are shown.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

The present invention includes a novel metabolomic analysis that is used to detect if a human subject with Alzheimer's Disease (AD) having an AD metabolomic phenotype and using the same for the treatment of the subject. Briefly, the method includes obtaining or having obtained a blood sample from the human subject with Alzheimer's Disease; measuring one or more levels of metabolites in the blood sample, wherein the metabolites are selected from at least one of benzenoids, organoheterocyclic compounds, prenol lipids, sterol lipids, fatty acyls, lipids and lipid-like molecules, cyclohexenones, imidazoles, and aryl alkyl ketones; applying an algorithm to the measured metabolite levels, the algorithm generating a metabolomic score based on a comparison of the measured metabolites levels to reference metabolites levels; identifying the human subject with AD as having an AD metabolomic phenotypebased on the metabolomic score; and based on the metabolomic score administering a drug treatment to the identified human subject; wherein the algorithm is selected from a machine learning algorithm, a clustering algorithm, a random forest algorithm, a support vector machines algorithm, a radial basis function algorithm and a combination thereof.

Examples of drug treatment based on the metabolomic score include, e.g., an antagonist of N-methyl-D-aspartate (NMDA) receptor subtype of the glutamate receptor, e.g., memantine, Cholinesterase inhibitors (ChEIs), e.g., donepezil, galantamine and rivastigmine, a monoclonal antibody against beta amyloid, lecanemab, anti-inflammatory drugs, NSA IDs, non-selective NSAIDs, selective NSAIDs, steroids, glucocorticoids, Immune Selective Anti-Inflammatory Derivatives (ImSAIDs), anti-TNF medications, anti-IL5 drugs, or CRP-lowering agents, and combinations thereof.

The present invention also includes ready-to-use kits for multiplex mass spectrometry analysis of AD blood metabolite biomarkers. The assay offers multiplexed tandem mass spectrometry (MS/MS) analysis of the two panels of metabolites (14 metabolites in Panel 1 and 20 metabolites in Panel 2) in only a 20-μL human serum sample. In one non-limiting example, the kit may contain a 96-well filter plate, reagents for sample preparation, blank and zero samples, multiple calibration standards (1, 2, 3, 4, 5, 6, 7, 8, 9, or 10), one or more levels of quality control (QC) samples (human serum-based QCs). The serum samples (20 μL), blank, zero sample, kit calibrator, and kit quality control material are each added directly onto the 96-well plate according to the predefined pipetting plan. After the proteins and salts are precipitated, the supernatants will be filtered into a lower sandwich plate by centrifugation at 200 g for 2 minutes. After a drying step of 30 minutes using nitrogen, the metabolite extracts are diluted for subsequent ultra-high performance liquid chromatography (UHPLC)-MS/MS analysis in selected reaction monitoring (SR M) mode or parallel reaction monitoring (PR M) as specified in the kit user manual.

The present invention can be used for different types and degrees of dementia including Alzheimer's Dementia. The present invention includes a substrate, such as a strip, that detects the combination of novel metabolites by simply dipping the strip in the serum sample or applying a drop of serum to the substrate.

The present invention also include substrate selected from at least one of: strip, particle, bead, biodegradable particle, sheet, gel, filter, membrane, nylon membrane, fiber, capillary, needle, microtiter strip, tube, plate, well, comb, pipette tip, micro array, chip, or slide.

The present invention used Support Vector Machine (SVM) algorithm with a Radial Basis Function (RBF) kernel, to identify metabolic features that predicted AD with a 0.0% classification error rate (1.00 Area Under the Curve (AUC) following 4-fold cross-validation). The computational model and biomarker set from the discovery phase, when tested against the verification dataset, retained their high predictive accuracy, yielding a 0.0% classification error rate (0.97 AUC).

The present inventor also used a Random Forest (RF) algorithm to identify additional metabolic features that predicted AD with a 0.0% classification error rate (1.00 AUC). The computational model and biomarker set from the discovery phase, when tested against the verification dataset, retained their high predictive accuracy, yielding a (0.96 AUC).

Thus, the present invention includes two panels of metabolic biomarkers formulated using two computational diagnostic models. The present invention is an efficient, blood-based diagnostic test for AD, for use in clinical screenings, diagnostic procedures, and treatments.

As taught herein, the inventors validated a set of metabolite biomarkers linked to AD diagnosis, using serum samples from the Texas Alzheimer's Research and Care Consortium (TARCC) (www.txalzresearch.org). It was found that blood-derived metabolites could distinguish AD from cognitively unimpaired controls. The present inventors used the precision of ultra-high-performance liquid chromatography-high resolution mass spectrometry (U PLC-HRMS) with advanced computational modeling techniques to achieve the present invention.

Ethical approval. This study was conducted under the approval of the University of Texas Medical Branch (UTMB) Institutional Review Board (IRB) protocol 21-0201 on Aug. 15, 2021. In addition, this study complied with all applicable federal regulations governing the protection of human subjects.

Cohort. Serum samples were requested through The Texas Alzheimer's Research and Care Consortium (TARCC) (www.txalzresearch.org) bio-banking facility. TARCC is a longitudinal collaborative research initiative between ten Texas medical research institutions.[26] TARCC aims to investigate factors involved in the development and progression of AD and improve early diagnosis, treatment, and prevention of Alzheimer's disease. The participants of the TARCC study were subjects older than 55 years and recruited at dementia clinics of the TARCC member institutions. All centers followed a harmonized collection protocol. The diagnosis of AD was based on the clinical examination with a neuropsychological battery using the National Institute of Neurological and Communicative Disorders and Stroke and the Alzheimer's Disease and Related Disorders Association (NINCDS-ADRDA) criteria[27], while age and sex-matched controls showed no cognitive impairment and achieved a zero on the Clinical Dementia Rating (CDR) scale.[28] The exclusion criteria include any significant neurological disease other than AD, such as Parkinson's disease, vascular dementia, progressive nonfluent aphasia, primary progressive aphasia, or prion disease. Participants with psychiatric disorders were also excluded. Age, sex, medical history, history of cardiovascular disease (i.e., heart disease, hypertension, diabetes mellitus, and hyperlipidemia), and family history of dementia were recorded.

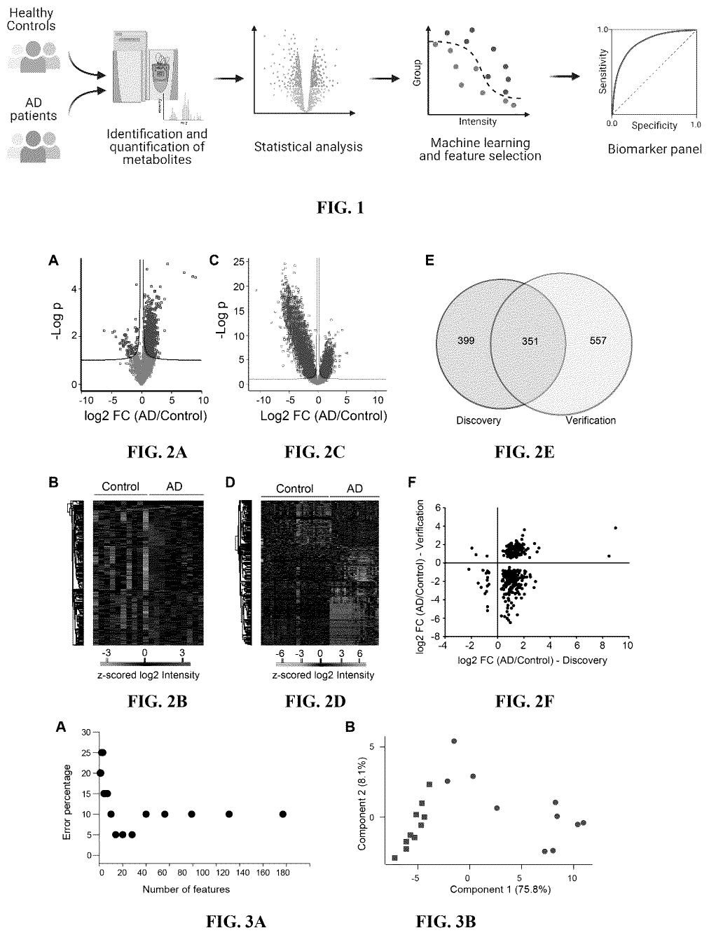

Study design. Serum samples from age and sex-matched AD patients and cognitively unimpaired older adults (Controls) were used. The study was conducted in two phases: the discovery phase and the verification phase. In the discovery phase, 20 serum samples were used (n=20; controls, 10; AD, 10). In the verification phase, 57 serum samples were used (n=57, controls, 30, AD, 27). The demographic data of the discovery and verification study are presented in Table 1. The workflow of biomarker discovery is shown in FIG. 1. Briefly, the serum metabolome of AD patients and cognitively unimpaired older adults were analyzed with LC-MS to identify AD-associated alternation in the metabolome. The significant metabolite hits were then used to identify a panel of AD biomarker candidates using machine learning technology. Finally, the performance of the biomarker panel was evaluated in the verification study.

TABLE 1
Demographic characteristics of the metabolomics studies.

| Demographic | Discovery Study | | | | Verification Study | | | |
|---|---|---|---|---|---|---|---|---|
| characteristics | Control (n = 10) | | AD (n = 10) | | Control (n = 30) | | AD (n = 27) | |
| Age, years (SD) | 68.60 | (7.12) | 73.00 | (11.37) | 68.40 | (6.50) | 69.19 | (9.86) |
| Sex, female (%) | 6 | (60.0%) | 4 | (40.0%) | 12 | (40.0%) | 10 | (37.0%) |
| CDR Global Score (SD) | 0.00 | (0.00) | 1.35 | (0.74) | 0.00 | (0.00) | 1.33 | (0.75) |
| Total MMSE score (SD) | 29.50 | (0.70) | 16.60 | (5.79) | 29.60 | (0.56) | 16.59 | (7.66) |
| Dx Probable AD (%) | 0 | (0.0%) | 10 | (100.0%) | 0 | (0.0%) | 27 | (100.0%) |
| Dx Frontotemporal | 0 | (0.0%) | 0 | (0.0%) | 0 | (0.0%) | 0 | (0.0%) |
| dementia (%) | | | | | | | | |
| Does subject meet criteria | 0 | (0.0%) | 10 | (100.0%) | 0 | (0.0%) | 27 | (100.0%) |
| for dementia? (%) | | | | | | | | |
| Does subject have normal | 10 | (100%) | 0 | (0.0%) | 30 | (100%) | 0 | (0.0%) |
| cognition? (%) | | | | | | | | |
| Systolic blood pressure | 124.30 | (9.91) | 132.33 | (13.84) | 125.83 | (9.92) | 131.31 | (13.84) |
| sitting (SD) | | | | | | | | |
| Hypercholesterolemia (%) | 5 | (50.0%) | 3 | (30.0%) | 12 | (40.0%) | 6 | (22.2%) |
| Hypertension (%) | 6 | (60.0%) | 5 | (50.0%) | 17 | (56.7%) | 10 | (37.0%) |
| Dx Other major | 0 | (0.0%) | 0 | (0.0%) | 0 | (0.0%) | 0 | (0.0%) |
| psychiatric illness (%) | | | | | | | | |
| Dx Parkinson's Disease | 0 | (0.0%) | 0 | (0.0%) | 0 | (0.0%) | 0 | (0.0%) |
| (%) | | | | | | | | |
| Dx Progressive nonfluent | 0 | (0.0%) | 0 | (0.0%) | 0 | (0.0%) | 0 | (0.0%) |
| aphasia (%) | | | | | | | | |
| Dx Possible vascular | 0 | (0.0%) | 0 | (0.0%) | 0 | (0.0%) | 0 | (0.0%) |
| dementia (%) | | | | | | | | |
| Dx Other disorder (%)** | 0 | (0.0%) | 0 | (0.0%) | 0 | (0.0%) | 0 | (0.0%) |
| Dx Primary progressive | 0 | (0.0%) | 0 | (0.0%) | 0 | (0.0%) | 0 | (0.0%) |
| aphasia (%) | | | | | | | | |
| Dx Prion disease (%) | 0 | (0.0%) | 0 | (0.0%) | 0 | (0.0%) | 0 | (0.0%) |
| A3_DADDEM (%) | 2 | (20.0%) | 5 | (50.0%) | 7 | (23.3%) | 10 | (37.0%) |
| A3_MOMDEM (%) | 4 | (40.0%) | 3 | (30.0%) | 15 | (50.0%) | 12 | (44.4%) |
| A3_SIBS (SD) | 2.50 | (1.84) | 1.70 | (1.63) | 0.17 | (1.68) | 1.67 | (1.35) |
| A3_SIBSDEM (%) | 0 | (0.0%) | 1 | (10.0%) | 1 | (3.3%) | 1 | (3.7%) |

CDR: Clinical Dementia Rating,
MMSE: Mini-mental State Exam,
AD: Alzheimer's Disease,
MCI: Mild Cognitive Impairment,
**Dx Other major psychiatric illness; logopenic, anomic, transcortical, word deafness, syntactic comprehension, or motor speech disorder,
A3_DADDEM: father with dementia, A3_MOMDEM: mother with dementia, A3_SIBS: number of siblings, A3_SIBSDEM: number of siblings with dementia.

CDR: Clinical Dementia Rating, MMSE: Mini-mental State Exam, AD: Alzheimer's Disease, MCI: Mild Cognitive Impairment, **Dx Other major psychiatric illness: Iogopenic, anomic, transcortical, word deafness, syntactic comprehension, or motor speech disorder, A3_DADDEM: father with dementia, A3_MM DEM: mother with dementia, A3_SIBS: number of siblings, A3_SIBSDEM: number of siblings with dementia.

Serum metabolome analysis with ultra-high-performance liquid chromatography-high resolution mass spectrometry. An aliquot of 20 μL of serum from each subject was deproteinized by acetonitrile precipitation (1:4 v:v). The deproteinized serum samples were dried using a nitrogen evaporator and resuspended in 0.1% formic acid for LC-MS/MS analysis. Samples were analyzed using an Easy-nLC 1000 UHPLC system coupled with a high-resolution, high-mass accuracy Q Exactive orbitrap mass spectrometer (Thermo Scientific). The metabolites were separated on a reverse-phase nano-HPLC column with a 20-min linear gradient from 2-95% mobile phase B (0.1% formic acid-90% acetonitrile) in mobile phase A (0.1% formic acid), followed by 30-min 90% mobile phase B. The Orbitrap was operated in data-dependent MS/MS mode with 70,000 resolution (FWHM) at the full scan and 17,500 (FWHM) at MS/MS. Each sample was analyzed two or three times.

Metabolomic data analysis. The MS data were analyzed with Compound Discoverer™ 3.3 software (Thermo Scientific) for metabolite identification and quantification. For metabolite identification, elemental compositions were predicted using accurate mass data, with compounds identified using the mzCloud mass spectral library and MS/MS information. mzCloud is a foremost mass spectral database that aids analysts in characterizing compounds even in cases where they may not be found in the library (www.mzcloud.org/). Where there was no match from mzCloud, ChemSpider was used. ChemSpider is a chemical structure database that is free of cost and allows connection to rapid text and structure probing of more than 100 million structures found in hundreds of sources of data (www.chemspider.com/). For results with a ChemSpider match, mzLogic was used to rank the identifications by the likelihood of a match. mzLogic is an algorithm for data analytics that merges the fragmentation data found inside the online mzCloud advanced mass spectral library with other data in various structural databases that are online. This permits you to choose thousands of structural possibilities and sort them numerically based on sub-structural data and spectral similarity (www.thermofisher.com/us/en/home/industrial/mass-spectrometry/liquid-chromatography-mass-spectrometry-lc-ms/lc-ms-software/mass-spectral-libraries/mzlogic-data-analysis.html). The metabolites identified with less than two structure annotation sources or mass error greater than five ppm were excluded from further analysis. The proposed identification results were then checked manually to eliminate false positives. Finally, the peak areas under the curve were extracted and integrated using the default setting of Compound Discoverer.

Statistical analysis. Statistical analyses were performed using Perseus Software 1.6.15.0.[29, 30] The discovery and verification datasets were analyzed independently. First, the intensity of each metabolite was log 2 transformed. Then the mean of log 2 transformed intensities of the technical replicates were calculated, and the group differences in metabolite MS intensity were analyzed using Student's t-test.

Hierarchical clustering and principal component analysis (PCA) were performed using Perseus Software 1.6.15.0.[29, 30] The unsupervised hierarchical clustering and heat map were based on the log 2-transformed MS intensity of metabolites. The rows of the heat map indicate the metabolites and the columns indicate the samples. The log 2 intensity of each metabolite was z-score normalized for each row. Hierarchical clustering of the z-normalized log 2 intensity was performed using Euclidean distances between means. The number of clusters was set at 300.

Classification analysis and feature selection.

Classification analysis and feature selection were performed using the machine learning module of Perseus Software 1.6.15.0.[29, 30] Support Vector Machine (SVM) modeling with Radial Basis Function (RBF) kernel was performed by 4-fold cross-validation. Duplicated features were removed based on feature optimization ranking prior to model building. Features that are medications and their metabolites were also excluded from model building. Measurements from the discovery cohort were used as the “training” data set, and those from the verification cohort as the “test” data set. Model performance was evaluated by analysis of the AUC of the ROC curve, where sensitivity (true positive) vs. 1-specificity (false positive) was plotted and assessment of classification accuracy.

In order to validate the analysis, intensities were standardized before the SVM model was run on the training dataset (Discovery cohort) with prefiltered metabolites, and predictions on the model using the test dataset (Verification cohort) were compared. To also look at individual metabolites, logistic regression models were fit for each one of the metabolites. Significant interactions between pairs of metabolites were searched using the rFSA algorithm.[31] Logistic regression analyses and SVM models were run on JMP version 16.1, SAS Institute Inc., Cary, NC. All other analyses were run using R studio version 2022.12.0 and R version 4.2.1. The significance for p-value was set at 0.05. Similarly, t-test with Permutation q-value<0.05 was used to control for false positive discovery rate in a multiple comparisons problem.

Study subjects. The discovery cohort participants comprised ten cognitively unimpaired older adults and ten AD patients (Table 1). The cognitively unimpaired older adults had a mean age of 68.60±7.12, a mean (Clinical Dementia Rating) CDR global score of 0.00±0.00, and a mean (Mini-Mental State Examination) MMSE score of 29.50±0.70. Six of the cognitively unimpaired older adults were women. The ten AD patients had a mean age of 73.00±11.37, a mean C D R global score of 1.35±0.74, and a mean MMSE score of 16.60±5.79. Four of the AD patients were women. The verification cohort participants comprised 30 cognitively unimpaired older adults and 27 AD patients (Table 1). The cognitively unimpaired older adults had a mean age of 68.40±6.50, a mean CDR global score of 0.00±0.00, and a mean MMSE score of 29.60±0.56. Twelve of the cognitively unimpaired older adults were women. The 27 AD patients had a mean age of 69.19±9.86, a mean CDR global score of 1.33±0.75, and a mean MMSE score of 16.59±7.66. Ten of the AD patients were women. N one of the discovery and verification cohort participants had any other types of dementia or other neurological diseases, such as Parkinson's disease.

Identification of blood-based metabolites that differentiate AD and cognitively unimpaired older adults.

Blood samples were analyzed using UHPLC-HRMS to identify metabolites that can distinguish between Alzheimer's patients and cognitively normal older adults. The results are as follows.

Discovery Phase (20 participants: 10 controls, 10 AD patients): 7,075 metabolites were detected. Of these, 1,414 showed significant differences between AD patients and controls (p-value<0.05) as indicated in FIGS. 2A, 2B. Some metabolites were detected multiple times in each sample. Using Support Vector algorithms, the inventors ranked them by their distinguishing ability and retained only the highest-ranking instance of each metabolite. This left 750 unique significant metabolites differentiating AD from cognitively unimpaired age matched older adults.

Verification Phase (57 participants: 30 controls, 27 AD patients): 10,595 metabolites were detected. 5,563 metabolites showed significant differences (Student's t-test, p-value<0.05). This was notably higher than in the discovery phase. To ensure robustness in the findings, the inventors adjusted for multiple comparisons using a Permutation-based method, aiming to maintain a false discovery rate (FD R) under 5%. This resulted in 5,136 significant metabolites, as shown in FIGS. 2C, 2D. After accounting for repeated metabolites, similar to the earlier process, 908 unique significant metabolites remained.

Comparison of Discovery and Verification Phases: 351 metabolites were found to be significant in both phases (FIG. 2E). Out of these, 162 metabolites had consistent differences (in fold change) between the AD and control groups across both studies. As shown in FIG. 2E, most of these 162 metabolites had consistent fold changes between the Discovery and Verification phases, but a few showed discrepancies. For instance, in the Discovery study, Hexylbenzene was found to be 504-fold more abundant in AD compared to healthy controls, whereas in the Verification study, it was only 13.86-fold higher. This may be due to the high variability of biomarker levels across the human population and the considerable molecular heterogeneity of individual AD patients. After excluding five metabolites related to medications, the final list consisted of 157 metabolites. These metabolites were then used to create computational diagnostic models for Alzheimer's Disease.

Classification feature optimization. Classification with Support Vector Machine algorithm.

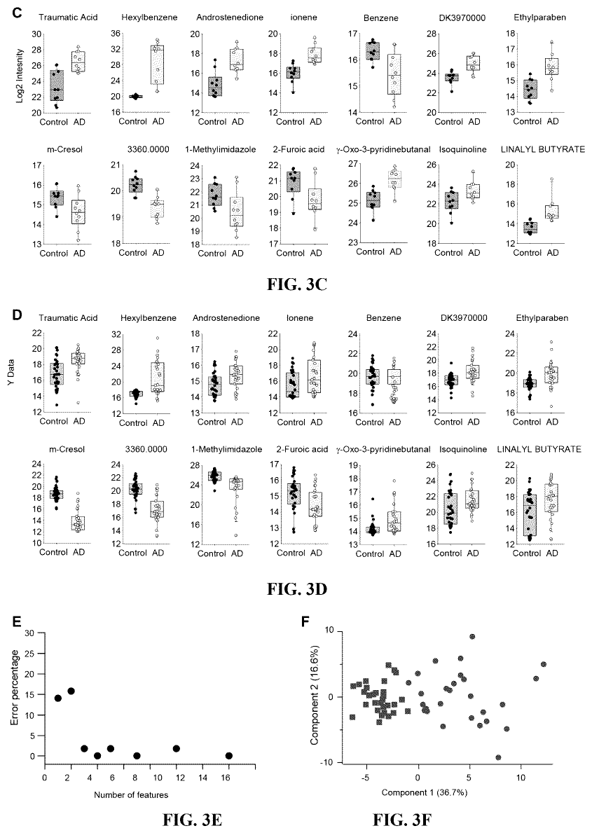

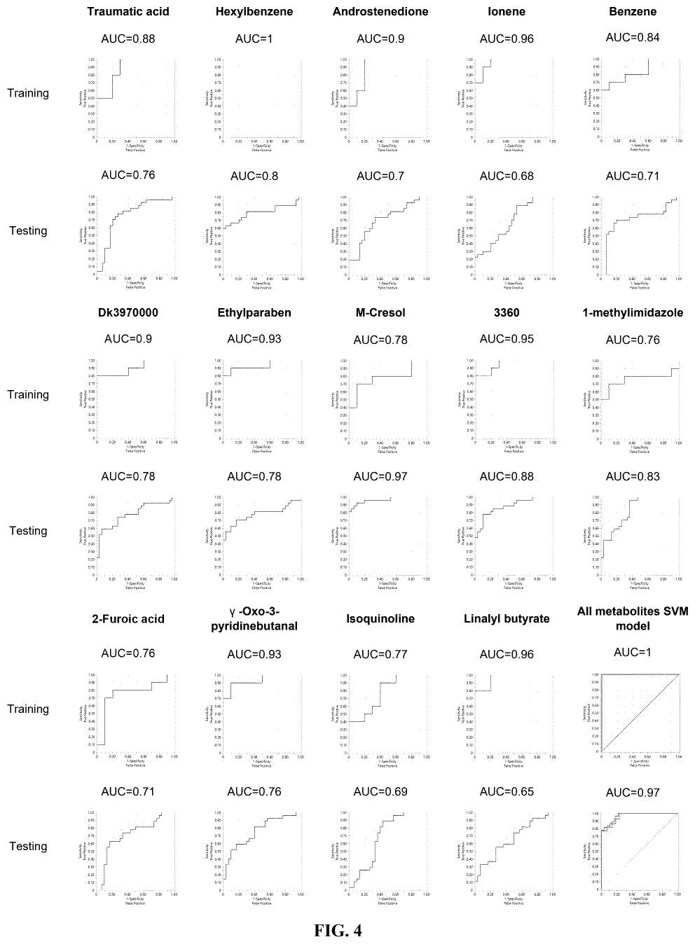

Support Vector Machine (SVM) algorithm with an RB F kernel was used, combined with 4-fold cross-validation, to rank the 157 metabolite features initially identified in the discovery study. The prediction error rate corresponding to the number of features utilized for AD prediction is depicted in FIG. 3A. Notably, employing the foremost 14 features resulted in the minimal error rate of 5%. Utilizing additional features did not enhance this rate. When principal component analysis (PCA) was performed on the abundances of these top 14 metabolites, the AD cohort was discernibly segregated from the cognitively unimpaired older group, as shown in FIG. 3. Hence, these 14 metabolites were chosen as potential biomarkers for AD diagnosis. Their abundance profiles in the serum of both AD patients and healthy older adults are presented in FIG. 3C and Table 2.

TABLE 2
The profiles of biomarker candidates of SVM model in the discovery and verification studies.

| | | | Discovery (n = 20, | | Verification (n = 57, | |
|---|---|---|---|---|---|---|
| | | | AD, 10; Control, 10) | | AD, 27; Control, 30) | |
| | | | Fold change | | Fold change | |
| Name | Formula | HMDB ID | (AD/Control) | p-value | (AD/Control) | p-value |
| Traumatic acid | C12H20O4 | HMDB0000933 | 8.82 | 0.00064 | 3.07 | 0.00072 |
| Hexylbenzene | C12H18 | HMDB0061815 | 504.21 | 0.00003 | 13.86 | 0.00002 |
| Androstenedione | C19H26O2 | HMDB0000053 | 5.38 | 0.00043 | 1.49 | 0.00741 |
| Ionene | C13H18 | HMDB0059826 | 3.64 | 0.00030 | 2.41 | 0.02019 |
| Benzene | C6H6 | HMDB0001505 | −1.85 | 0.00615 | −2.18 | 0.00341 |
| Dk3970000 | C14H20O2 | HMDB0013817 | 2.60 | 0.00080 | 2.56 | 0.00048 |
| Ethylparaben | C9H10O3 | HMDB0032573 | −0.38 | 0.00052 | −0.49 | 0.00037 |
| M-Cresol | C7H8O | HMDB0002048 | −1.71 | 0.02414 | −26.95 | 0.00000 |
| 3360 | C7H10O | HMDB0031541 | −1.81 | 0.00007 | −8.56 | 0.00000 |
| 1-methylimidazole | C4H6N2 | HMDB0243940 | −2.40 | 0.03449 | −6.33 | 0.00017 |
| 2-Furoic acid | C5H4O3 | HMDB0000617 | −2.18 | 0.02694 | −1.76 | 0.00594 |
| γ-Oxo-3- | C9H9NO2 | HMDB0004186 | 2.07 | 0.00033 | 1.71 | 0.00138 |
| pyridinebutanal | | | | | | |
| Isoquinoline | C9H7N | HMDB0034244 | 2.18 | 0.02462 | 2.11 | 0.03542 |
| Linalyl butyrate | C14H24O2 | HMDB0030427 | 3.22 | 0.00138 | 2.65 | 0.03340 |

In the verification phase, the inventors assessed if these 14 metabolites could differentiate the AD cohort from the cognitively unimpaired older adults. Their profiles in this phase are illustrated in FIG. 3D and Table 2, revealing a congruence with the discovery phase profiles. The prediction error in the verification study, utilizing these 14 metabolite profiles and SVM with an RBF kernel combined with 4-fold cross-validation, was 0.0%, as visualized in FIG. 3E. A PCA based on these 14 metabolites' abundances distinctly segregated the AD cohort from the cognitively healthy older adults, as displayed in FIG. 3F.

Further, using the discovery dataset, a model was trained and subsequently tested it on the verification dataset. The efficacy of the resulting models in disease differentiation was evaluated using ROC curves, which graphically represent sensitivity against (1-specificity) for variable discrimination thresholds.[32] The AUC signifies a model's discriminative capacity between outcome groups. The ROCs for individual metabolites from both datasets are shown in FIG. 4. In the discovery dataset, 10 out of the 14 metabolites attained an AUC exceeding 0.8 for AD prediction, whereas in the verification dataset, only four metabolites achieved this threshold. Incorporating all 14 metabolites considerably augmented model efficacy. The AUC values were 1.00 for the training dataset and 0.97 for the testing dataset, showcasing a robust performance in AD prediction. The confusion matrices for the models across both datasets can be found in Table 3, highlighting an accuracy rate of 100% for the training dataset and 86% for the testing dataset. Additionally, the inventors investigated potential interaction terms for synergistic effects. Significant interactions were discerned between Hexylbenzene and Ethylparaben (p-value=0.031), Traumatic Acid and 2,6-Di-tert-butylbenzoquinone (Dk3970000) (p-value=0.01), and benzene and 3360 (p-value=0.026).

TABLE 3
Confusion matrices for the model in both datasets.

| Training Dataset | | | | Testing Dataset | | | |
|---|---|---|---|---|---|---|---|
| | | Predicted | | | | Predicted | |
| | Total | Control | AD | | Total | Control | AD |
| | n = 20 | n = 10 | n = 10 | | n = 57 | n = 30 | n = 27 |
| Ac- | Con- | 10 | 0 | Ac- | Con- | 26 | 4 |
| tual | trol 10 | | | tual | trol 30 | | |
| | AD 10 | 0 | 10 | | AD 27 | 4 | 23 |

Associations of metabolites with gender. The inventors stratified the AD group and cognitively unimpaired older adults group (control) by sex. As shown in Table 4, in the female group, nine out of the 14 metabolites significantly differed between AD and cognitively unimpaired older adults, and in the male group, 11 out of the 14 metabolites significantly differed between AD and cognitively unimpaired older adults. Some of the metabolite biomarkers were not found to be significant in each gender group, probably due to the reduced sample size. In the group of cognitively unimpaired older adults, the abundance of the 14 metabolites shows no significant difference between females and males. However, in the AD group, the level of androstenedione in male AD patients was significantly higher than in female AD patients. Taken together, androstenedione is the only biomarker within the biomarker panel that correlates with gender.

TABLE 4
Association of 14 metabolite biomarkers with
gender. The t-test p-values < 0.05 was bolded.

| | Females | | Males | | Controls | | AD | |
|---|---|---|---|---|---|---|---|---|
| | AD (n = 10) vs. | | AD (n = 17) vs. | | Female (n = 12) | | Female (n = 10) | |
| | Control (n = 12) | | Control (n = 18) | | vs. Male (n = 18) | | vs. Male (n = 17) | |
| | Fold | p- | Fold | p- | Fold | p- | Fold | p- |
| Metabolites | change | value | change | value | change | value | change | value |
| Traumatic acid | 3.87 | 0.007 | 2.64 | 0.032 | −0.45 | 0.524 | 0.10 | 0.872 |
| Hexylbenzene | 13.63 | 0.025 | 13.97 | <0.001 | −0.09 | 0.778 | −0.13 | 0.944 |
| Androstenedione | 1.16 | 0.463 | 1.72 | 0.006 | −0.18 | 0.52 | −0.74 | 0.023 |
| Ionene | 1.30 | 0.642 | 3.51 | 0.014 | 0.41 | 0.514 | −1.02 | 0.280 |
| Benzene | −3.28 | 0.002 | −1.71 | 0.136 | 0.07 | 0.88 | −0.87 | 0.151 |
| Dk3970000 | 2.80 | 0.008 | 2.40 | 0.019 | −0.41 | 0.29 | −0.19 | 0.784 |
| Ethylparaben | 1.76 | 0.122 | 2.21 | 0.001 | 0.03 | 0.902 | −0.30 | 0.579 |
| M-Cresol | −38.56 | <0.001 | −21.69 | <0.001 | 0.19 | 0.708 | −0.65 | 0.409 |
| 3360 | −7.53 | 0.002 | −9.38 | <0.001 | −0.49 | 0.384 | −0.17 | 0.838 |
| 1-methylimidazole | −14.44 | 0.011 | −3.94 | 0.002 | −0.29 | 0.457 | −2.17 | 0.116 |
| 2-Furoic acid | 0.69 | 0.238 | −1.97 | 0.014 | −0.14 | 0.747 | 0.30 | 0.482 |
| γ-Oxo-3- | 1.41 | 0.070 | 1.91 | 0.008 | −0.18 | 0.39 | −0.62 | 0.173 |
| pyridinebutanal | | | | | | | | |
| Isoquinoline | 2.06 | 0.226 | 2.16 | 0.090 | 0.34 | 0.679 | 0.28 | 0.657 |
| Linalyl butyrate | 2.35 | 0.219 | 2.79 | 0.095 | −0.65 | 0.494 | −0.90 | 0.351 |

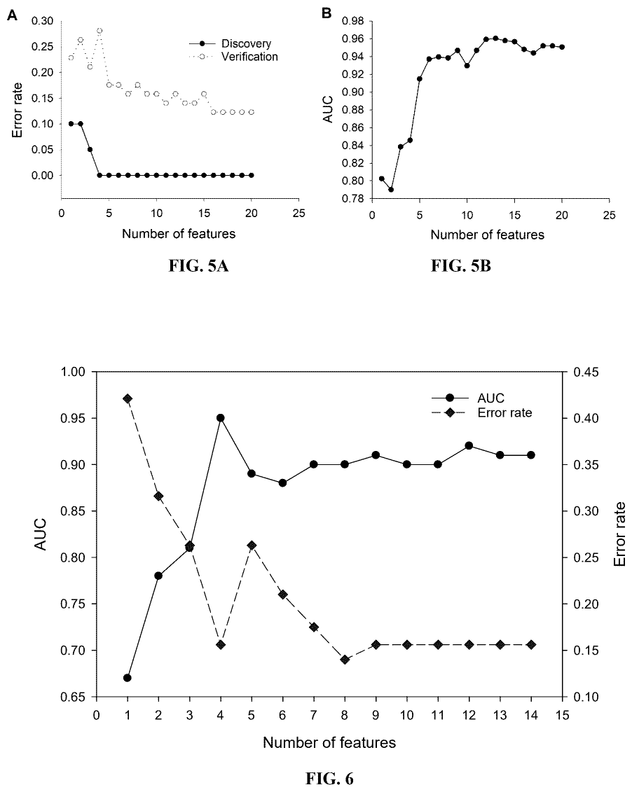

Classification with Random Forest. The inventors used the discovery phase dataset to apply Breiman and Cutler's RF algorithm to analyze 157 metabolite features identified in the discovery and verification studies and identified 20 features based on importance. Table 5 shows the profiles of these 20 metabolites in the serum of AD patients and cognitively unimpaired older adults and their importance in the RF model. The inventors examined the prediction error using the profiles of 20 metabolites. As shown in FIG. 5A, in the discovery study, the model yielded 0.0% prediction error rate with top five features and increasing the number of features did not change the error rate. The prediction error rate for the verification study was higher than the discovery rate. The error rate with the top five features was 17.5% and the lowest error rate was 12.3% with top 16 features. Next, the model was trained using the discovery dataset and tested the trained model with the verification dataset. In this analysis, the performance of the resulting models in disease classification was examined using ROC curves. FIG. 5B shows the AUC of ROCs. As the number of features increased, the AUC improved and the model with 13 features yielded the highest AUC of 0.96. Among the 13 features, six are overlapped with the features of SVM model, including Hexylbenzene, 3-Methyl-2-cyclohexen-1-one, γ-Oxo-3-pyridinebutanal, DK3970000, Androstenedione. The principal component analysis (PCA) using the abundance of the top 13 metabolites shows that the AD group was distinctly separated from cognitively unimpaired older adults (FIGS. 5C, 5D).

Table 5. The profiles of biomarker candidates of RF model in the discovery and verification studies.

TABLE 5
The profiles of biomarker candidates of RF model in the discovery and verification studies.

| | | | Discovery (n = 20, | | Verification (n = 57, | | |
|---|---|---|---|---|---|---|---|
| | | | AD, 10; Control, 10) | | AD, 27; Control, 30) | | |
| | | | Fold change | | Fold change | | |
| Name | Formula | HMDB ID | (AD/Control) | p-value | (AD/Control) | p-value | Importance |
| Hexylbenzene* | C12H18 | HMDB0061815 | 504.21 | 0.00003 | 13.86 | 0.00002 | 0.798396 |
| N-Nonanoylglycine | C11H21NO3 | HMDB0013279 | 2.69 | 0.00303 | 1.69 | 0.02339 | 0.37687 |
| Ionene* | C13H18 | HMDB0059826 | 3.64 | 0.00030 | 2.41 | 0.02019 | 0.29151 |
| 2401/delta- | C12H22O2 | HMDB0037116 | 3.58 | 0.00168 | 2.45 | 0.00641 | 0.289255 |
| Dodecalactone | | | | | | | |
| 3360/3-Methyl- | C7H10O | HMDB0031541 | −1.81 | 0.00007 | −8.56 | 0.00000 | 0.289138 |
| 2-cyclohexen-1-one* | | | | | | | |
| (5Z,8Z)-5,8- | C14H24O2 | HMDB0340799 | 3.44 | 0.02998 | 1.85 | 0.00173 | 0.257661 |
| Tetradecadienoic | | | | | | | |
| acid | | | | | | | |
| FG7175000 | C9H7NO | | 2.69 | 0.00175 | 1.55 | 0.00498 | 0.212159 |
| γ-Oxo-3- | C9H9NO2 | HMDB0004186 | 1.10 | 0.00033 | 1.71 | 0.00138 | 0.211026 |
| pyridinebutanal* | | | | | | | |
| DK 3970000/2,6- | C14H20O2 | HMDB0013817 | 2.60 | 0.00080 | 2.56 | 0.00048 | 0.201963 |
| Di-tert- | | | | | | | |
| butylbenzoquinone* | | | | | | | |
| 3,4-Dimethyl-5- | C14H22O3 | | 3.99 | 0.00723 | 3.98 | 0.00004 | 0.164183 |
| pentyl-2- | | | | | | | |
| furanpropanoic | | | | | | | |
| acid | | | | | | | |
| Androstenedione* | C19H26O2 | HMDB0000053 | 5.38 | 0.00043 | 1.49 | 0.00741 | 0.15991 |
| DL-carvone | C10H14O | HMDB0035089 | 1.93 | 0.00543 | 2.73 | 0.00000 | 0.157633 |
| Decanoic acid | C10H20O2 | HMDB0000511 | 3.05 | 0.00370 | 2.65 | 0.00001 | 0.140389 |
| 10-Undecenoic | C11H20O2 | HMDB0033724 | 2.91 | 0.01305 | 2.72 | 0.00508 | 0.138158 |
| acid | | | | | | | |
| MFCD00009868 | C16H28O2 | HMDB0340889 | 3.32 | 0.00355 | 2.96 | 0.00004 | 0.134775 |
| 4-Vinylcyclohexene | C8H12 | HMDB0246592 | −1.95 | 0.00185 | −10.11 | 0.00019 | 0.131989 |
| 1-Phenyl-2- | C12H16O | HMDB0094661 | 2.00 | 0.00132 | 3.19 | 0.00013 | 0.130808 |
| hexanone | | | | | | | |
| 2781/Gamma- | C9H16O2 | HMDB0031531 | 2.24 | 0.00642 | 2.16 | 0.00853 | 0.129112 |
| nonalactone | | | | | | | |
| 2,2- | C9H8O2 | HMDB0032947 | 2.82 | 0.00237 | 1.86 | 0.00002 | 0.121297 |
| Methylenebisfuran | | | | | | | |
| Allylcyclohexane | C9H16 | HMDB0061920 | 2.14 | 0.00217 | 2.75 | 0.00309 | 0.12 |

*Features that overlapped with the features in SVM model.

Based on the chemical structures, the metabolites features in SVM and RE models can be grouped into benzenoids (benzene; m-cresol; ethylparaben, hexylbenzene, ionene), organoheterocyclic compounds (2-furoic acid; isoquinoline), prenol lipids (2,6-di-tert-butylbenzoquinone, 2,6-D i-tert-butylbenzoquinone; (R)-Carvone), sterol lipids (androstenedione), fatty acyls (traumatic acid, gamma-nonalactone; Decanoic acid; Undecylenic acid), lipids and lipid-like molecules (Linalyl butyrate), cyclohexenones (3-Methyl-2-cyclohexen-1-one), imidazoles (1-methylimidazole), and aryl alkyl ketones (gamma-Oxo-3-pyridinebutanal).

Peripheral blood tests for AD have emerged as a more convenient alternative, especially for repeated sampling in clinical trials.[6] However, current blood-based tests, even those that assess Tau and β-amyloid levels, are limited by the scope of metabolites they can evaluate. As such, there is a need for expanded paradigms of blood diagnostics for AD. The present inventors used machine learning to discover novel blood biomarkers that differentiate AD from cognitively unimpaired older adults with high specificity and sensitivity, laying the foundation for developing a blood diagnostic for AD. Thus, the inventors used an integrated high-resolution mass spectrometry (HRMS)-based metabolomics technology and machine learning algorithms for developing biomarker identification and classification model.

HRMS is one of the most commonly used technologies in metabolomics studies and offers excellent sensitivity and accuracy in metabolite identification. The inventors' data-driven approach trained algorithms with labeled samples, each represented by a set of features, and the resulting classification model was able to predict the label of a sample from its features.

As used herein, the labels correspond to AD patients and cognitively unimpaired older adults, and the features are the abundance of metabolites in serum. The biomarker candidates that differentiated AD from cognitively unimpaired older adults were first identified using a discovery cohort, then these candidates were verified with a much larger verification cohort. In both discovery and verification studies, the dataset acquired in the discovery study was fed to the supervised machine-learning algorithms, and the resulting classification models were tested with the verification dataset. Because the number of significant metabolite features identified in the discovery and verification studies was larger than that which warrants further investigation, the inventors performed feature reduction as commonly used in classification model development to narrow the list of potential biomarkers down to a more reasonable number by removing noisy data, redundant variables, or low-reproducible data.

The resulting 157 significant metabolite features were used for supervised machine learning. Both SVM and RF were performed, and each classifier selected a panel of biomarker candidates that accurately differentiated AD from cognitively unimpaired older adults. In comparison to newly discovered blood biomarkers for AD classification, the 14-metabolite SVM model or 13-metabolites RF model disclosed herein are comparably accurate to the plasma p-tau181 (AUC=0.85-0.98)^{76,77}, plasma p-tau217 (AUC=0.89-0.96)⁷⁹, a 19-protein panel (AUC=0.96-0.98)⁸⁰, and better than a 5-metabolite panel (AUC=0.84)⁸¹, and outperformed a 5-metabolite panel (AUC=0.84)⁸¹ and a 2-metablites+age panel (AUC=0.72)⁸¹. Importantly, the inventors rigorously validated the biomarker panels identified in this study using an independent set of samples and confirmed their excellent specificity and sensitivity in diagnosing AD.

Some of metabolic features identified by SVM and RF algorithms are overlapped, including Hexylbenzene, ionene, 3-Methyl-2-cyclohexen-1-one, γ-Oxo-3-pyridinebutanal, DK3970000, Androstenedione. Several studies previously identified potential plasma or CSF AD metabolic biomarkers classified as benzene and substituted derivatives.[44-46] However, none of this study's five benzenoid biomarkers have been reported previously. Among the five benzenoids, Hexylbenzene has the best predictive power, which AUC of ROCs in the Discovery and Verification studies are 1.0 and 0.8, respectively. Hexylbenzene has been identified from healthy human body fluids.[47] The results herein show that hexylbenzene was significantly elevated in the serum of AD patients.

Androstenedione is a steroidal hormone that is naturally occurring and made in the gonads and adrenal glands of both sexes. It is the common precursor of male and female sex hormones and can be transformed into testosterone and estrogen depending on the tissue type. The inventors found that androstenedione was elevated in the serum of AD patients. Interestingly, significant sex-differences were found in the correlation between androstenedione level with cognitive capacity. In the female group, the abundance of androstenedione shows no significant difference between AD and cognitively unimpaired aged adults. In the male group, androstenedione level was significantly elevated compared to cognitively unimpaired aged adults. In addition, in the AD group, the level of androstenedione in male AD patients was significantly higher than in female AD patients. Vankovi et al. also found that the level of androstenedione in AD patients was elevated compared to healthy controls, but they found that androstenedione was higher in women with AD than controls but not in men.[48] This inconsistency may be because the level of androstenedione is sex and age-dependent, and there is a significant age difference between AD patients and healthy controls (73.7 vs. 66.4) in Vankovi's study.

1-Methylimidazole was found to be lower in AD serum and a significant metabolite candidate in the SVM model of the discovery and verification study (Table 2; Table 4, Row 10). Recently, a link between 1-Methylimidazole and AD pathology was proposed but not shown. The connection points to how amyloid-β peptide (Aβ) binds copper (II) with high affinity in its N-terminal region.[50] This is in line with earlier studies that reported the presence of Aβ-Cu interactions in the formation of toxic oligomers[51, 52], but proved no correlation.

The inventors also identified isoquinoline to be higher in AD serum and to be a significant metabolite in the SVM model (Table 2; Table 4, Row 13). Protoberberines are the most studied of eight subgroups of isoquinoline alkaloids (IAs), where cholinesterase activity relevant to AD has been identified.

5,8 tetradecadienoic acid (Table 5, Row 6), was found to be a biomarker candidate in the RF model. An earlier study identified a panel of five plasma metabolites that allowed to differentiate between amnestic mild cognitive impairment (aMCI) and control subjects, but not in AD. The panel included 5,8 tetradecadienoic acid together with thymine, arachidonic acid, 2-aminoadipic acid, and N,N-dimethylglycine.

The inventors also identified decanoic acid as a biomarker candidate following the RF model analysis (Table 5, Row 13). The inventors found decanoic acid to be an associated biomarker candidate in the RF model for the discovery and verification studies.

The RF model also found 10-Undecenoic acid (undecylenic acid), a naturally occurring unsaturated fatty acid, to be a candidate biomarker (Table 5, Row 14).

4-Vinylcyclohexene (VCH) was another significant biomarker found in the RF model (Table 5, Row 16). Studies have often used VCH and the 4-4-Vinylcyclohexene diepoxide metabolite (VCD) for induction of ovarian failure in mice. Importantly, both occupational chemicals have been identified as having carcinogenic potential based on studies conducted in mice and rats. Consequently, it was found that VCH and VCD caused ovarian follicle and uterine atrophy in mice exposed for 13 weeks.[65]

Traumatic acid, a fatty acid derivative, was first isolated and synthesized by English, Bonner, and Haagen-Smit in 1939.[68] It is a known wound hormone which causes parenchymatous tissues in plants (bean pods and potato tubers) to proliferate.[69] Recently, Traumatic acid was tested in cytotoxicity assessments in which it was found that it may act as a compound that delays the stimulatory effect of pesticides on breast cancer cells.[69, 70] As shown herein, the biological significance of Traumatic acid could be diet related.

Classification with endogenous metabolites.

Some of the biomarkers in Tables 2 and 5 are either exclusive to plants or are not naturally occurring (exposome). The presence of these metabolites in human blood is due to diet or environmental exposure. Previous research has demonstrated that exogenous metabolites and environmental exposures play a substantial role in AD⁷¹⁻⁷⁶. This underscores the importance of identifying, quantifying, and characterizing the entire spectrum of endogenous and exogenous metabolites in AD biological specimen samples and examining the biochemical processes linked to AD onset and evolution. Next, the inventors created an effective computational model for diagnosing AD using only endogenous metabolites. Metabolites were excluded that are exclusively from plants or are not naturally occurring based on the Human Metabolome Database (HMBD) ontology section (hmdb.ca). The remaining 56 endogenous metabolites were used for computational modeling, with the discovery dataset serving as the training dataset and the verification dataset as the testing dataset. Initially, the random forest algorithm was used to assess the importance of each metabolite. Subsequently, the top 14 metabolites (Table 6) were analyzed using the SVM algorithm. As shown in FIG. 6, increasing the number of features improves the area under the curve (AUC) of receiver operating characteristics (ROC) and reduces the prediction error rate. The model with four features -N-Nonanoylglycine, delta-Dodecalactone, 2,6-Di-tert-butylbenzoquinone, and 3-Methyl-2-cyclohexen-1-one- exhibits the best AUC.

TABLE 6
The profiles of biomarker candidates of SVM model using endogenous metabolites

| | | | Discovery (n = 20, | | Verification (n = 57, | | |
|---|---|---|---|---|---|---|---|
| | | | AD, 10; Control, 10) | | AD, 27; Control, 30) | | |
| | | | Fold change | | Fold change | | |
| Name | Formula | HMDB ID | (AD/Control) | p-value | (AD/Control) | p-value | Rank |
| N-Nonanoylglycine | C11H21NO3 | HMDB0013279 | 2.69 | 0.003 | 1.69 | 0.023 | 1 |
| delta-Dodecalactone | C12H22O2 | HMDB0037116 | 3.58 | 0.002 | 2.45 | 0.006 | 2 |
| 2,6-Di-tert- | C14H20O2 | HMDB0013817 | 2.60 | <0.001 | 2.56 | <0.001 | 3 |
| butylbenzoquinone | | | | | | | |
| 3-Methyl-2- | C7H10O | HMDB0031541 | −1.81 | <0.001 | −8.56 | <0.001 | 4 |
| cyclohexen-1-one | | | | | | | |
| Linalyl Butyrate | C14H24O2 | HMDB0030427 | 3.22 | 0.001 | 2.65 | 0.033 | 5 |
| γ-Oxo-3- | C9H9NO2 | HMDB0004186 | 2.07 | <0.001 | 1.71 | 0.001 | 6 |
| pyridinebutanal | | | | | | | |
| LILAC | C9H10O | HMDB0029639 | 1.81 | 0.002 | 3.18 | <0.001 | 7 |
| ACETALDEHYDE | | | | | | | |
| Traumatic Acid | C12H20O4 | HMDB0000933 | 8.82 | <0.001 | 3.07 | <0.001 | 8 |
| Homovanillin | C9H10O3 | HMDB0005175 | 2.69 | 0.01 | 1.72 | 0.004 | 9 |
| 2-Tridecenal | C13H24O | HMDB0037275 | 2.70 | 0.008 | 2.55 | 0.022 | 10 |
| Androstenedione | C19H26O2 | HMDB0000053 | 5.38 | <0.001 | 1.49 | 0.007 | 11 |
| 2-Acetyl-3,5- | C8H10O2 | HMDB0032159 | 1.78 | 0.01 | 2.80 | <0.001 | 12 |
| dimethylfuran | | | | | | | |
| 3-oxopalmitic acid | C16H30O3 | HMDB0010733 | 2.71 | 0.004 | 1.54 | 0.02 | 13 |
| 8-Hydroxy-5,6- | C8H12O3 | HMDB0031101 | 2.67 | 0.02 | 1.55 | 0.01 | 14 |
| octadienoic acid | | | | | | | |

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

An AD metabolomic score of the present invention may be calculated with an algorithm described herein. Specific examples of statistical analysis techniques that may be used to calculate the AD metabolomic score are described hereinabove. In addition, one or more of the following may also be used, including, cross-correlation, Principal Components Analysis (PCA), factor rotation, Logistic Regression (LogReg), Linear Discriminant Analysis (LDA), Eigengene Linear Discriminant Analysis (ELDA), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, and Hidden Markov Models, Linear Regression or classification algorithms, Nonlinear Regression or classification algorithms, analysis of variants (A NOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms.

In preferred embodiments, the risk score may be calculated using a random forest algorithm using the concentrations of three or more sample analytes in the panel of biomarkers. In an exemplary embodiment, the risk score is calculated as described in the examples.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (RO M) for storing software, random access memory (RAM), and nonvolatile storage. Other hardware, conventional and/or custom, may also be included.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Knopman D S, Amieva H, Petersen R C, et al. Alzheimer disease. Nat Rev Dis Primers. 2021; 7(1):33.
2. Wu Y T, Beiser A S, Breteler M M B, et al. The changing prevalence and incidence of dementia overtime—current evidence. Nat Rev Neurol. 2017; 13(6):327-339.
3. Long J M, Holtzman D M. Alzheimer Disease: An Update on Pathobiology and Treatment Strategies. Cell. 2019; 179(2):312-339.
4. Jack C R, Jr., Bennett D A, Blennow K, et al. A/T/N: An unbiased descriptive classification scheme for Alzheimer disease biomarkers. Neurology. 2016; 87(5):539-547.
5. Jack C R, Jr., Bennett D A, Blennow K, et al. NIA-A A Research Framework: Toward a biological definition of Alzheimer's disease. Alzheimers Dement. 2018; 14(4):535-562.
6. Mattsson N, Carrillo M C, Dean R A, et al. Revolutionizing Alzheimer's disease and clinical trials through biomarkers. Alzheimers Dement (Amst). 2015; 1(4):412-419.
7. Blennow K, Zetterberg H. Biomarkers for Alzheimer's disease: current status and prospects for the future. J Intern M ed. 2018; 284(6):643-663.
8. Lashley T, Schott J M, Weston P, et al. Molecular biomarkers of Alzheimer's disease: progress and prospects. Dis Model Mech. 2018; 11(5).
9. Simren J, Leuzy A, Karikari T K, et al. The diagnostic and prognostic capabilities of plasma biomarkers in Alzheimer's disease. Alzheimers Dement. 2021; 17(7):1145-1156.
10. Cullen N C, Leuzy A, J anelidze S, et al. Plasma biomarkers of Alzheimer's disease improve prediction of cognitive decline in cognitively unimpaired elderly populations. Nat Commun. 2021; 12(1):3555.
11. Voyle N, Kim M, Proitsi P, et al. Blood metabolite markers of neocortical amyloid-beta burden: discovery and enrichment using candidate proteins. Transl Psychiatry. 2016; 6:e719.
12. Zhang X, Alshakhshir N, Zhao L. Glycolytic Metabolism, Brain Resilience, and Alzheimer's Disease. Front Neurosci. 2021; 15:662242.
13. Yassine H N, Finch C E. APOE Alleles and Diet in Brain Aging and Alzheimer's Disease. Front Aging Neurosci. 2020; 12:150.
14. de Leon M J, Convit A, Wolf O T, et al. Prediction of cognitive decline in normal elderly subjects with 2-[(18)F]fluoro-2-deoxy-D-glucose/poitron-emission tomography (FDG/PET). Proc Natl Acad Sci USA. 2001; 98(19):10966-10971.
15. Mosconi L, DeSanti S, Li J, et al. Hippocampal hypometabolism predicts cognitive decline from normal aging. Neurobiol Aging. 2008; 29(5):676-692.
16. Wieckowska-Gacek A, Mietelska-Porowska A, Wydrych M, Wojda U. Western diet as a trigger of Alzheimer's disease: From metabolic syndrome and systemic inflammation to neuroinflammation and neurodegeneration. Ageing Res Rev. 2021; 70:101397.
17. Pillai J A, Bena J, Bekris L, et al. Metabolic syndrome biomarkers relate to rate of cognitive decline in MCI and dementia stages of Alzheimer's disease. Alzheimers Res Ther. 2023; 15(1):54.
18. Ng T P, Feng L, Nyunt M S, et al. Metabolic Syndrome and the Risk of Mild Cognitive Impairment and Progression to Dementia: Follow-up of the Singapore Longitudinal Ageing Study Cohort. JAMA Neurol. 2016; 73(4):456-463.
19. Sabbagh M N, Perez A, Holland™, et al. Primary prevention recommendations to reduce the risk of cognitive decline. Alzheimers Dement. 2022; 18(8):1569-1579.
20. Horgusluoglu E, Neff R, Song W M, et al. Integrative metabolomics-genomics approach reveals key metabolic pathways and regulators of Alzheimer's disease. Alzheimers Dement. 2022; 18(6):1260-1278.
21. Schumacher-Schuh A, Bieger A, Borelli W V, Portley M K, Awad P S, Bandres-Ciga S. Advances in Proteomic and Metabolomic Profiling of Neurodegenerative Diseases. Front Neurol. 2021; 12:792227.
22. Jia L, Y ang J, Zhu M, et al. A metabolite panel that differentiates Alzheimer's disease from other dementia types. Alzheimers Dement. 2022; 18(7):1345-1356.
23. Pappolla M A, Perry G, Fang X, Zagorski M, Sambamurti K, Poeggeler B. Indoles as essential mediators in the gut-brain axis. Their role in Alzheimer's disease. Neurobiol Dis. 2021; 156:105403.
24. Kalecky K, German D C, Montillo A A, Bottiglieri T. Targeted Metabolomic Analysis in Alzheimer's Disease Plasma and Brain Tissue in Non-Hispanic Whites. J Alzheimers Dis. 2022; 86(4):1875-1895.
25. Teruya T, Chen Y J, Kondoh H, Fukuji Y, Yanagida M. Whole-blood metabolomics of dementia patients reveal classes of disease-linked metabolites. Proc Natl Acad Sci USA. 2021; 118(37).
26. Waring S, O'Bryant S, Reisch J, Diaz-Arrastia R, Knebl J, Doody R. The Texas Alzheimer's Research Consortium longitudinal research cohort: Study design and baseline characteristics. Texas Public H ealth J. 2008; 60:9-13.
27. McKhann G, Drachman D, Folstein M, Katzman R, Price D, Stadlan E M. Clinical diagnosis of Alzheimer's disease: report of the NINCDS-ADRDA Work Group under the auspices of Department of Health and Human Services Task Force on Alzheimer's Disease. Neurology. 1984; 34(7):939-944.
28. Hughes C P, Berg L, Danziger W L, Coben L A, Martin R L. A new clinical scale for the staging of dementia. Br J Psychiatry. 1982; 140:566-572.
29. Tyanova S, Cox J. Perseus: A Bioinformatics Platform for Integrative Analysis of Proteomics Data in Cancer Research. Methods Mol Biol. 2018; 1711:133-148.
30. Tyanova S, Temu T, Sinitcyn P, et al. The Perseus computational platform for comprehensive analysis of (prote)omics data. Nat Methods. 2016; 13(9):731-740.
31. Lambert J, Gong L, Elliott C F, Thompson K, Stromberg A. rFSA: An R Package for Finding Best Subsets and Interactions. R J. 2018; 10(2):295-308.
32. Zweig M H, Campbell G. Receiver-operating characteristic (ROC) plots: a fundamental evaluation tool in clinical medicine. Clinical chemistry. 1993; 39(4):561-577.
33. Heiles S. Advanced tandem mass spectrometry in metabolomics and lipidomics-methods and applications. Anal Bioanal Chem. 2021; 413(24):5927-5948.
34. Letertre M P M, Dervilly G, Giraudeau P. Combined Nuclear Magnetic Resonance Spectroscopy and Mass Spectrometry Approaches for Metabolomics. Anal Chem. 2021; 93(1):500-518.
35. Massetti N, Russo M, Franciotti R, et al. A Machine Learning-Based Holistic Approach to Predict the Clinical Course of Patients within the Alzheimer's Disease Spectrum. J Alzheimers Dis. 2022; 85(4):1639-1655.
36. Franciotti R, Nardini D, Russo M, et al. Comparison of Machine Learning-based Approaches to Predict the Conversion to Alzheimer's Disease from Mild Cognitive Impairment. Neuroscience. 2023; 514:143-152.
37. Wang Z, Y e J, Zhang K, et al. Rapid Biomarker Screening of Alzheimer's Disease by Interpretable Machine Learning and Graphene-Assisted Raman Spectroscopy. ACS Nano. 2022; 16(4):6426-6436.
38. Spratt H M, J u H. Statistical Approaches to Candidate Biomarker Panel Selection. Advances in experimental medicine and biology. 2016; 919:463-492.
39. Karikari T K, Pascoal T A, Ashton N J, et al. Blood phosphorylated tau 181 as a biomarker for Alzheimer's disease: a diagnostic performance and prediction modelling study using data from four prospective cohorts. Lancet Neurol. 2020; 19(5):422-433.
40. Karikari T K, Benedet A L, Ashton N J, et al. Diagnostic performance and prediction of clinical progression of plasma phospho-tau181 in the Alzheimer's Disease Neuroimaging Initiative. Mol Psychiatry. 2021; 26(2):429-442.
41. Palmqvist S, Janelidze S, Quiroz Y T, et al. Discriminative Accuracy of Plasma Phospho-tau217 for Alzheimer Disease vs Other Neurodegenerative Disorders. JAMA. 2020; 324(8):772-781.
42. Jiang Y, Zhou X, Ip F C, et al. Large-scale plasma proteomic profiling identifies a high-performance biomarker panel for Alzheimer's disease screening and staging. Alzheimers Dement. 2022; 18(1):88-102.
43. Shao Y, Ouyang Y, Li T, et al. Alteration of Metabolic Profile and Potential Biomarkers in the Plasma of Alzheimer's Disease. Aging Dis. 2020; 11(6):1459-1470.
44. Kaddurah-Daouk R, Zhu H, Sharma S, et al. Alterations in metabolic pathways and networks in Alzheimer's disease. Transl Psychiatry. 2013; 3(4):e244.
45. Trushina E, Dutta T, Persson X M, Mielke M M, Petersen R C. Identification of altered metabolic pathways in plasma and CSF in mild cognitive impairment and Alzheimer's disease using metabolomics. PLoS One. 2013; 8(5):e63644.
46. Gonzalez-Dominguez R, Garcia-Barrera T, Gomez-Ariza J L. Application of a novel metabolomic approach based on atmospheric pressure photoionization mass spectrometry using flow injection analysis for the study of Alzheimer's disease. Talanta. 2015; 131:480-489.
47. de Lacy Costello B, Amann A, Al-Kateb H, et al. A review of the volatiles from the healthy human body. J Breath Res. 2014; 8(1):014001.
48. Vankova M, Velikova M, Vejrazkova D, et al. The Role of Steroidomics in the Diagnosis of Alzheimer's Disease and Type 2 Diabetes Mellitus. Int J Mol Sci. 2023; 24(10).
49. Camp B W, Gerson J E, Tsang C W, et al. High serum androstenedione levels correlate with impaired memory in the surgically menopausal rat: a replication and new findings. Eur J Neurosci. 2012; 36(8):3086-3095.
50. Atrian-Blasco E, Gonzalez P, Santoro A, Alies B, Faller P, Hureau C. Cu and Zn coordination to amyloid peptides: From fascinating chemistry to debated pathological relevance. Coord Chem Rev. 2018; 375:38-55.
51. Savelieff M G, Lee S, Liu Y, Lim M H. Untangling amyloid-beta, tau, and metals in Alzheimer's disease. ACS Chem Biol. 2013; 8(5):856-865.
52. Rana M, Sharma A K. Cu and Zn interactions with A beta peptides: consequence of coordination on aggregation and formation of neurotoxic soluble Abeta oligomers. Metallomics. 2019; 11(1):64-84.
53. Carvalho A, Barbosa B M, Flores J S, et al. New mescaline-related N-acylhydrazone and its unsubstituted benzoyl derivative: Promising metallophores for copper-associated deleterious effects relief in Alzheimer's disease. J Inorg Biochem. 2023; 238:112033.
54. Cahlikova L, Vrabec R, Pidany F, et al. Recent Progress on Biological Activity of Amaryllidaceae and Further Isoquinoline Alkaloids in Connection with Alzheimer's Disease. Molecules. 2021; 26(17).
55. Honorio P, Sainimnuan S, Hannongbua S, Saparpakorn P. Binding interaction of protoberberine alkaloids against acetylcholinesterase (AChE) using molecular dynamics simulations and QM/MM calculations. Chem Biol Interact. 2021; 344:109523.
56. Liu P, Chen X, Zhou H, et al. The Isoquinoline A Ikaloid Dauricine Targets Multiple Molecular Pathways to Ameliorate Alzheimer-Like Pathological Changes In Vitro. Oxid M ed Cell Longev. 2018; 2018:2025914.
57. Wang G, Zhou Y, Huang F J, et al. Plasma metabolite profiles of Alzheimer's disease and mild cognitive impairment. J Proteome Res. 2014; 13(5):2649-2658.
58. Ameen A O, Freude K, Aldana Bl. Fats, Friends or Foes: Investigating the Role of Short- and Medium-Chain Fatty Acids in Alzheimer's Disease. Biomedicines. 2022; 10(11).
59. Rebello C J, Keller J N, Liu A G, Johnson W D, Greenway F L. Pilot feasibility and safety study examining the effect of medium chain triglyceride supplementation in subjects with mild cognitive impairment: A randomized controlled trial. BBA Clin. 2015; 3:123-125.
60. Avgerinos K I, Egan J M, Mattson M P, Kapogiannis D. Medium Chain Triglycerides induce mild ketosis and may improve cognition in Alzheimer's disease. A systematic review and meta-analysis of human studies. Ageing Res Rev. 2020; 58:101001.
61. Fortier M, Castellano C A, Croteau E, et al. A ketogenic drink improves brain energy and some measures of cognition in mild cognitive impairment. Alzheimers Dement. 2019; 15(5):625-634.
62. Mett J, Muller U. The medium-chain fatty acid decanoic acid reduces oxidative stress levels in neuroblastoma cells. Sci Rep. 2021; 11(1):6135.
63. Mett J. The Impact of Medium Chain and Polyunsaturated omega-3-Fatty Acids on Amyloid-beta Deposition, Oxidative Stress and Metabolic Dysfunction Associated with Alzheimer's Disease. Antioxidants (Basel). 2021; 10(12).
64. Lee E, Eom J E, Kim H L, et al. Neuroprotective effect of undecylenic acid extracted from Ricinus communis L. through inhibition of mu-calpain. Eur J Pharm Sci. 2012; 46(1-2):17-25.
65. Kappeler C J, Hoyer P B. 4-vinylcyclohexene diepoxide: a model chemical for ovotoxicity. Syst Biol Reprod M ed. 2012; 58(1):57-62.
66. Pontifex M G, Martinsen A, Saleh R N M, et al. A POE4 genotype exacerbates the impact of menopause on cognition and synaptic plasticity in APOE-T R mice. FASEB J. 2021; 35(5):e21583.
67. Pontifex M G, Martinsen A, Saleh R N M, et al. DHA-Enriched Fish Oil Ameliorates Deficits in Cognition Associated with Menopause and the APOE4 Genotype in Rodents. Nutrients. 2022; 14(9).
68. English J, Jr., Bonner J, Haagen-Smit A J. Structure and Synthesis of a Plant Wound Hormone. Science. 1939; 90(2336):329.
69. Farmer E E. Fatty acid signalling in plants and their associated microorganisms. Plant Mol Biol. 1994; 26(5):1423-1437.
70. Jablonska-Trypuc A, Wydro U, Wolejko E, Butarewicz A. Toxicological Effects of Traumatic Acid and Selected Herbicides on Human Breast Cancer Cells: In Vitro Cytotoxicity Assessment of Analyzed Compounds. Molecules. 2019; 24(9).
71. Finch C E and Kulminski A M. The Alzheimer's Disease Exposome. Alzheimers Dement 2019; 15: 1123-1132. DOI: 10.1016/j.jalz.2019.06.3914.
72. Lefevre-Arbogast S, Chaker J, Mercier F, et al. Assessing the contribution of the chemical exposome to neurodegenerative disease. Nat Neurosci 2024; 27: 812-821. DOI: 10.1038/s41593-024-01627-1.
73. Sakowski S A, Koubek E J, Chen K S, et al. Role of the Exposome in Neurodegenerative Disease: Recent Insights and Future Directions. Ann Neurol 2024; 95: 635-652. DOI: 10.1002/ana.26897.
74. Vardarajan B, Kalia V, Manly J, et al. Differences in plasma metabolites related to Alzheimer's disease, A POE epsilon4 status, and ethnicity. Alzheimers Dement (N Y) 2020; 6: e12025. DOI: 10.1002/trc2.12025.
75. Vignoli A and Tenori L. N M R-based metabolomics in Alzheimer's disease research: a review. Front Mol Biosci 2023; 10: 1308500. DOI: 10.3389/fmolb.2023.1308500.
76. Wang F, Chen D, Wu P, et al. Formaldehyde, Epigenetics, and Alzheimer's Disease. Chem Res Toxicol 2019; 32: 820-830. DOI: 10.1021/acs.chemrestox.9b00090.
77. Karikari T K, Pascoal T A, Ashton N J, et al. Blood phosphorylated tau 181 as a biomarker for Alzheimer's disease: a diagnostic performance and prediction modelling study using data from four prospective cohorts. Lancet Neurol 2020; 19: 422-433. DOI: 10.1016/51474-4422(20)30071-5.
78. Karikari T K, Benedet A L, Ashton N J, et al. Diagnostic performance and prediction of clinical progression of plasma phospho-tau181 in the Alzheimer's Disease Neuroimaging Initiative. Mol Psychiatry 2021; 26: 429-442. DOI: 10.1038/s41380-020-00923-z.
79. Palmqvist S, Janelidze S, Quiroz Y T, et al. Discriminative Accuracy of Plasma Phospho-tau217 for Alzheimer Disease vs Other Neurodegenerative Disorders. JAMA 2020; 324: 772-781. DOI: 10.1001/jama.2020.12134.
80. Jiang Y, Zhou X, Ip F C, et al. Large-scale plasma proteomic profiling identifies a high-performance biomarker panel for Alzheimer's disease screening and staging.
Alzheimers Dement 2022; 18: 88-102. DOI: 10.1002/alz.12369.
81. Shao Y, Ouyang Y, Li T, et al. Alteration of Metabolic Profile and Potential Biomarkers in the Plasma of Alzheimer's Disease. Aging Dis 2020; 11: 1459-1470. DOI: 10.14336/AD.2020.0217.
82. Lin C N, Huang C C, Huang K L, et al. A metabolomic approach to identifying biomarkers in blood of Alzheimer's disease. Ann Clin Transl Neurol 2019; 6: 537-545. 20190227. DOI: 10.1002/acn3.726.

What is claimed is:

1. A method for treating a human subject with Alzheimer's Disease (AD) having an AD metabolomic phenotype, the method comprising:
obtaining or having obtained a blood sample from the human subject with AD;
measuring one or more levels of metabolites in the blood sample, wherein the metabolites are selected from at least one of benzenoids, organoheterocyclic compounds, prenol lipids, sterol lipids, fatty acyls, lipids and lipid-like molecules, cyclohexenones, imidazoles, and aryl alkyl ketones;
applying an algorithm to the one or more measured metabolite levels, the algorithm generating a metabolomic score based on a comparison of the one or more measured metabolites levels to reference metabolites levels; and
identifying the human subject with AD as having an AD metabolomic phenotype based on the metabolomic score;
wherein the algorithm is selected from a machine learning algorithm, a clustering algorithm, a random forest algorithm, a support vector machines algorithm, a radial basis function algorithm and a combination thereof; and
administering to the human subject with the AD metabolomic score one or more agents that slow progression of AD.

2. The method of claim 1, wherein the machine learning algorithm is selected from at least one of: Random Forest, Support Vector Machines, and Radial Basis Function.

3. The method of claim 1, wherein the one or more agents is selected from at least one of:
an antagonist of N-methyl-D-aspartate (NMDA) receptor subtype of the glutamate receptor, memantine, Cholinesterase inhibitors (ChEIs), donepezil, galantamine and rivastigmine, a monoclonal antibody against beta amyloid, lecanemab, anti-inflammatory drugs, NSAIDs, non-selective NSAIDs, selective NSA IDs, steroids, glucocorticoids, Immune Selective Anti-Inflammatory Derivatives (ImSAIDs), anti-TNF medications, anti-IL5 drugs, CRP-lowering agents, anti-apoptotic compounds, metal chelators, inhibitors of DNA repair, 3-amino-1-propanesulfonic acid (3APS), 1,3-propanedisulfonate (1,3PDS), secretase activators, beta- and gamma-secretase inhibitors, neurotransmitters, beta-sheet breakers, anti-inflammatory molecules, antipsychotics, cholinesterase inhibitor, a corticosteroid, an antibiotic, an antiviral agent, an anti-Tau antibody, semorinemab, BMS-986168, C2N-8E12, Gosuranemab, Tilavonemab, Zagotenemab, a Tau inhibitor, a Tau N-terminal binder, a Tau mid-domain binder, a fibrillar Tau binder, an anti-amyloid-beta (anti-Ap) antibody, bapineuzumab, solanezumab, aducanumab, gantenerumab, crenezumab, donanembab, lecanemab, an beta-amyloid aggregation inhibitor, an anti-BACE1 antibody, a BACE1 inhibitor, a monoamine depletory agent, an ergoloid mesylate, an anticholinergic antiparkinsonism agent, a dopaminergic antiparkinsonism agent, a tetrabenazine, a dimebolin, a homotaurine, or a serotonin receptor activity modulator.

4. The method of claim 1, wherein the blood sample is a whole blood or a plasma sample, and wherein one or more biomarkers are measured by at least one method selected from ultra-high-performance liquid chromatography-high resolution mass spectrometry (U PLC-HRMS), an immunoassay, an enzymatic activity assay, fluorescence detection, chemiluminescence detection, electrochemiluminescence detection and patterned array, antibody binding, fluorescence activated sorting, detectable bead sorting, antibody array, microarray, enzymatic array, receptor binding array, solid-phase binding array, liquid phase binding array, fluorescent resonance transfer, and radioactive labeling.

5. The method of claim 1, wherein the algorithm is selected from Support Vector Machine (SVM) and Random Forest (RF) algorithms.

6. The method of claim 1, wherein the algorithm is selected from Support Vector Machine (SVM) with a Radial Basis Function (RBF) kernel.

7. The method of claim 1, wherein the at least one of:
benzenoids are selected from at least one of: benzene, m-cresol, ethylparaben, hexylbenzene, or ionene;
N-Nonanoylglycine, delta-Dodecalactone, 2,6-Di-tert-butylbenzoquinone, and 3-Methyl-2-cyclohexen-1-one;
the organoheterocyclic compounds are selected from at least one of: 2-furoic acid or isoquinoline;
the prenol lipids are selected from at least one of: 2,6-di-tert-butylbenzoquinone, 2,6-Di-tert-butylbenzoquinone; or (R)-Carvone;
the sterol lipid is androstenedione;
the fatty acyls are selected from at least one of: traumatic acid, gamma-nonalactone; Decanoic acid; or Undecylenic acid;
the lipids and lipid-like molecules, cyclohexenones, imidazoles and aryl alkyl ketones are selected from at least one of: Linalyl butyrate, 3-Methyl-2-cyclohexen-1-one, 1-methylimidazole, and gamma-Oxo-3-pyridinebutanal, respectively;
the metabolites are selected from Hexylbenzene, Ionene, 3-Methyl-2-cyclohexen-1-one, γ-Oxo-3-pyridinebutanal, 2,6-Di-tert-butylbenzoquinone (DK3970000), and Androstenedione; or
the metabolites are selected from at least one of: Traumatic acid, Hexylbenzene, Benzene, 2,6-D i-tert-butyl benzoquinone (DK3970000), Ethylparaben, M-Cresol, 3-Methyl-2-cyclohexen-1-one (3360), 1-methylimidazole, or γ-Oxo-3-pyridinebutanal.

8. The method of claim 1, wherein the metabolites are selected in the following order: N-nonanoylglycine, delta-dodecalactone, 2,6-di-tert-butylbenzoquinone, 3-methyl-2-cyclohexen-1-one, linalyl butyrate, γ-oxo-3-pyridinebutanal, lilac acetaldehyde, traumatic acid, homovanillin, 2-tridecenal, androstenedione, 2-acetyl-3,5-dimethylfuran, 3-oxopalmitic acid, and 8-hydroxy-5,6-octadienoic acid.

9. A method for treating a human subject with Alzheimer's Disease (AD) having an AD metabolomic phenotype, the method comprising:
obtaining or having obtained a blood sample from the human subject with Alzheimer's disease;
measuring one or more levels of metabolites in the blood sample, wherein the metabolites are selected from 1, 2, 3, 4, 5, 6, 7, 8, 9, 10, 11, 12, 13, 14, 15, 16, 17, 18, 19, or 20 metabolites selected from Hexylbenzene, N-Nonanoylglycine, Ionene, 2401/delta-Dodecalacton, 3360/3-Methyl-2-cyclohexen-1-one, (5Z,8Z)-5,8-Tetradecadienoic acid, FG7175000, γ-Oxo-3-pyridinebutanal, DK3970000/2,6-Di-tert-butylbenzoquinone, 3,4-Dimethyl-5-pentyl-2-furanpropanoic acid, Androstenedione, DL-carvone, Decanoic acid, 10-Undecenoic acid, M FCD00009868, 4-Vinylcyclohexene, 1-Phenyl-2-hexanone, 2781/Gamma-nonalactone, 2,2-Methylenebisfuran, Allylcyclohexane;
applying an algorithm to the one or more measured metabolite levels, the algorithm generating a metabolomic score based on a comparison of the measured metabolites levels to reference metabolites levels;
identifying the human subject with Alzheimer's Disease as having an AD metabolomic phenotype based on the metabolomic score;
wherein the algorithm is selected from a machine learning algorithm, a clustering algorithm, a random forest algorithm, a support vector machines algorithm, a radial basis function algorithm and a combination thereof.

10. The method of claim 9, wherein the machine learning algorithm is selected from at least one of: Random Forest, Support Vector Machines, and Radial Basis Function.

11. The method of claim 9, further comprising administering a drug treatment to the human subject identified with the metabolomic score, wherein the drug treatment is selected from at least one of: an antagonist of N-methyl-D-aspartate (NMDA) receptor subtype of the glutamate receptor, memantine, Cholinesterase inhibitors (ChEIs), donepezil, galantamine and rivastigmine, a monoclonal antibody against beta amyloid, lecanemab, anti-inflammatory drugs, NSAIDs, non-selective NSAIDs, selective NSAIDs, steroids, glucocorticoids, Immune Selective Anti-Inflammatory Derivatives (ImSAIDs), anti-TNF medications, anti-IL5 drugs, C-reactive protein (CRP)-lowering agents, anti-apoptotic compounds, metal chelators, inhibitors of DNA repair, 3-amino-1-propanesulfonic acid (3APS), 1,3-propanedisulfonate (1,3PDS), secretase activators, beta- and gamma-secretase inhibitors, neurotransmitters, beta-sheet breakers, anti-inflammatory molecules, antipsychotics, cholinesterase inhibitor, a corticosteroid, an antibiotic, an antiviral agent, an anti-Tau antibody, semorinemab, BMS-986168, C2N-8E12, Gosuranemab, Tilavonemab, Zagotenemab, a Tau inhibitor, a Tau N-terminal binder, a Tau mid-domain binder, a fibrillar Tau binder, an anti-amyloid-beta (anti-Aβ) antibody, bapineuzumab, solanezumab, aducanumab, gantenerumab, crenezumab, donanembab, lecanemab, an beta-amyloid aggregation inhibitor, an anti-BACE1 antibody, a BACE inhibitor, a monoamine depletory agent, an ergoloid mesylate, an anticholinergic antiparkinsonism agent, a dopaminergic antiparkinsonism agent, a tetrabenazine, a dimebolin, a homotaurine, or a serotonin receptor activity modulator.

12. The method of claim 9, wherein the blood sample is a whole blood or a plasma sample, and wherein one or more biomarkers are measured by at least one method selected from ultra-high-performance liquid chromatography-high resolution mass spectrometry (UPLC-HRMS), an immunoassay, an enzymatic activity assay, fluorescence detection, chemiluminescence detection, electrochemiluminescence detection and patterned array, antibody binding, fluorescence activated sorting, detectable bead sorting, antibody array, microarray, enzymatic array, receptor binding array, solid-phase binding array, liquid phase binding array, fluorescent resonance transfer, and radioactive labeling.

13. The method of claim 9, wherein the algorithm is selected from Support Vector Machine (SVM) and Random Forest (RF) algorithms or Support Vector Machine (SVM) with a Radial Basis Function (RBF) kernel.

14. The method of claim 9, wherein the metabolites are selected in the following order: N-nonanoylglycine, delta-dodecalactone, 2,6-di-tert-butylbenzoquinone, 3-methyl-2-cyclohexen-1-one, linalyl butyrate, γ-oxo-3-pyridinebutanal, lilac acetaldehyde, traumatic acid, homovanillin, 2-tridecenal, androstenedione, 2-acetyl-3,5-dimethylfuran, 3-oxopalmitic acid, and 8-hydroxy-5,6-octadienoic acid.

15. A method for treating a human subject with Alzheimer's Disease (AD) having an AD metabolomic phenotype, the method comprising:
obtaining or having obtained a blood sample from the human subject with Alzheimer's disease;
measuring a level of one or more metabolites in the blood sample, wherein the one or more metabolites are selected from 1, 2, 3, 4, 5, or 6 metabolites selected from N-nonanoylglycine, delta-dodecalactone, 2,6-di-tert-butylbenzoquinone, 3-methyl-2-cyclohexen-1-one, linalyl butyrate, or γ-oxo-3-pyridinebutanal;
applying an algorithm to the one or more measured metabolite levels, the algorithm generating a metabolomic score based on a comparison of the measured metabolites levels to reference metabolites levels;
identifying the human subject with Alzheimer's Disease as having an AD metabolomic phenotype based on the metabolomic score; and
administering an anti-inflammatory a drug treatment to the identified human subject;
wherein the algorithm is selected from a machine learning algorithm, a clustering algorithm, a random forest algorithm, a support vector machines algorithm, a radial basis function algorithm and a combination thereof; and
wherein the drug treatment is selected from at least one of: an antagonist of N-methyl-D-aspartate (NMDA) receptor subtype of the glutamate receptor, memantine, Cholinesterase inhibitors (ChEIs), donepezil, galantamine, rivastigmine, a monoclonal antibody against beta amyloid, lecanemab, anti-inflammatory drugs, NSAIDs, non-selective NSAIDs, selective NSAIDs, steroids, glucocorticoids, Immune Selective Anti-Inflammatory Derivatives (ImSAIDs), anti-TN F medications, anti-IL5 drugs, C-reactive protein (CRP)-lowering agents, anti-apoptotic compounds, metal chelators, inhibitors of DNA repair, 3-amino-1-propanesulfonic acid (3APS), 1,3-propanedisulfonate (1,3PDS), secretase activators, beta- and gamma-secretase inhibitors, neurotransmitters, beta-sheet breakers, anti-inflammatory molecules, antipsychotics, cholinesterase inhibitor, a corticosteroid, an antibiotic, an antiviral agent, an anti-Tau antibody, semorinemab, BMS-986168, C2N-8E12, Gosuranemab, Tilavonemab, Zagotenemab, a Tau inhibitor, a Tau N-terminal binder, a Tau mid-domain binder, a fibrillar Tau binder, an anti-amyloid-beta (anti-Ap) antibody, bapineuzumab, solanezumab, aducanumab, gantenerumab, crenezumab, donanembab, lecanemab, an beta-amyloid aggregation inhibitor, an anti-BACE1 antibody, a BACE1 inhibitor, a monoamine depletory agent, an ergoloid mesylate, an anticholinergic antiparkinsonism agent, a dopaminergic antiparkinsonism agent, a tetrabenazine, a dimebolin, a homotaurine, or a serotonin receptor activity modulator.

16. The method of claim 15, further comprising measuring one or more additional level of metabolites in the blood sample selected from at least one of: (5Z,8Z)-5,8-Tetradecadienoic acid, FG7175000, 3,4-Dimethyl-5-pentyl-2-furanpropanoic acid, DL-carvone, Decanoic acid, 10-Undecenoic acid, M FCD00009868, 4-Vinylcyclohexene, 1-Phenyl-2-hexanone, 2781/Gamma-nonalactone, 2,2-Methylenebisfuran, or Allylcyclohexane, Hexylbenzene, Ionene, and Androstenedione.

17. A kit for determining an Alzheimer's Disease (AD) metabolomic phenotype, the kit comprising:
a substrate comprising reagents that binds to one or more metabolites selected from 1, 2, 3, 4, 5, or 6 metabolites selected from N-nonanoylglycine, delta-dodecalactone, 2,6-di-tert-butylbenzoquinone, 3-methyl-2-cyclohexen-1-one, linalyl butyrate, or γ-oxo-3-pyridinebutanal, wherein the substrate is adapted for quantitative metabolite analysis; and
instructions for the quantitative metabolite analysis.

18. The kit of claim 17, further comprising additional reagents measuring one or more additional level of metabolites in a blood sample selected from at least one of: (5Z,8Z)-5,8-Tetradecadienoic acid, FG7175000, 3,4-Dimethyl-5-pentyl-2-furanpropanoic acid, DL-carvone, Decanoic acid, 10-Undecenoic acid, MFCD00009868, 4-Vinylcyclohexene, 1-Phenyl-2-hexanone, 2781/Gamma-nonalactone, 2,2-Methylenebisfuran, or Allylcyclohexane, Hexylbenzene, Ionene, and Androstenedione.

19. The kit of claim 17, wherein the substrate is selected for use in multiplexed tandem mass spectrometry (MS/MS) or high-performance liquid chromatography (UHPLC)-MS/MS analysis.

20. The kit of claim 17, wherein the substrate is at least one of: strip, particle, bead, biodegradable particle, sheet, gel, filter, membrane, nylon membrane, fiber, capillary, needle, microtiter strip, tube, plate, well, comb, pipette tip, micro array, chip, or slide.

21. A non-transitory computer-readable medium for determining an Alzheimer's Disease (AD) metabolomic score in a subject comprising instructions stored thereon, that when executed on a processor, perform the steps of:
receiving an electronic communication containing data for one or more levels of metabolites in a blood sample, wherein the metabolites are selected from at least one of benzenoids, organoheterocyclic compounds, prenol lipids, sterol lipids, fatty acyls, lipids and lipid-like molecules, cyclohexenones, imidazoles, and aryl alkyl ketones of the subject;
using a processor to process an algorithm to the data for one or more levels of metabolites, the algorithm generating a metabolomic score based on a comparison of the data for one or more levels of metabolites to reference metabolites levels, wherein the algorithm is selected from a machine learning algorithm, a clustering algorithm, a random forest algorithm, a support vector machines algorithm, a radial basis function algorithm and a combination thereof;
identifying the human subject with Alzheimer's Disease as having an AD metabolomic phenotype based on the metabolomic score; and
administering to the human subject with the AD metabolomic score one or more agents that slow progression of AD.

22. A computer-implemented method for determining a personalized risk assessment for a subject, the method comprising:
receiving an electronic communication containing data for one or more levels of metabolites in the blood sample, wherein the metabolites are selected from at least one of benzenoids, organoheterocyclic compounds, prenol lipids, sterol lipids, fatty acyls, lipids and lipid-like molecules, cyclohexenones, imidazoles, and aryl alkyl ketones of the subject;
using a processor to process an algorithm to the data for one or more levels of metabolites, the algorithm generating a metabolomic score based on a comparison of the data for one or more levels of metabolites to reference metabolites levels, wherein the algorithm is selected from a machine learning algorithm, a clustering algorithm, a random forest algorithm, a support vector machines algorithm, a radial basis function algorithm and a combination thereof;
identifying that the subject has Alzheimer's Disease and as having an AD metabolomic phenotype based on the metabolomic score; and
administering to the human subject with the AD metabolomic score one or more agents that slow progression of AD.
